(19) United States
(12) Patent Application Publication
(10) Pub. No.: US 2019/0151660 A1
(43) Pub. Date: May 23, 2019

(54) SYSTEMS AND METHODS FOR TREATMENT OF PAIN FOLLOWING AMPUTATION AND SURGICAL TISSUE REMOVAL

(71) Applicant: SPR Therapeutics, Inc., Cleveland, OH (US)

(72) Inventors: Joseph W. Boggs, II, Chapel Hill, NC (US); Nathan D. Crosby, Cleveland, OH (US); Amorn Wongsarnpigoon, Chapel Hill, NC (US)

(21) Appl. No.: 16/036,992

(22) Filed: Jul. 17, 2018

Related U.S. Application Data
(60) Provisional application No. 62/589,043, filed on Nov. 21, 2017.

(51) Int. Cl. A61N 1/36

(52) U.S. Cl. CPC A61N 1/36021; A61N 1/0456; A61N 1/36017

(57) ABSTRACT

Pain felt in a given region of the body can be treated by stimulating a peripheral nerve at a therapeutically effective distance from the region where pain is felt to generate a comfortable sensation (i.e., paresthesia) overlapping the regions of pain. Systems and methods to reduce pain in a painful region following surgical amputation of an extremity or limb may include stimulating a peripheral nerve innervating the painful region with an electrode inserted into tissue and spaced from the peripheral nerve. This method may be used to help alleviate postoperative pain in patients following amputation of an extremity or limb.

### CROSS-REFERENCE TO RELATED APPLICATIONS

This application claims priority to, and all benefit, from U.S. Provisional Patent Application No. 62/589,043 filed on Nov. 21, 2017, which is incorporated herein by reference in its entirety.

### TECHNICAL FIELD

The present disclosure generally relates to a system and method to deliver electrical stimulation to treat pain; and more particularly, to a system and method to deliver electrical stimulation to treat pain following a procedure to amputate or remove a limb, extremity, or body part or portion thereof.

### BACKGROUND

Amputation can become necessary for a variety of reasons including, but not limited to, trauma, cancer, infection, birth defect, or vascular disease. Amputation procedures often result in significant post-operative pain that can cause a delay in rehabilitation and impact the patient's ability to use a prosthesis or return to activities of daily living. Post-amputation pain may include pain in the residual limb, also called the stump, and/or pain in the phantom limb, which is the experience of sensations in a representation of the portion of the limb that is no longer present. While existing systems and techniques offer some relief and ancillary benefits to individuals requiring therapeutic relief, many issues with such systems exist. Therefore, there is a need for an improved system and method.

Amputees commonly experience acute and chronic post-amputation pain. Amputation leads to persistent pain in 70-90% of patients. This pain results in decreased quality of life, increased risk of depression, negative impacts on interpersonal relationships and negatively affects the ability to work. Both acute and persistent post-amputation pain are commonly managed with opioids that are associated with undesirable adverse effects such as nausea, vomiting, sedation, and respiratory depression. Other treatments for post-amputation pain may include non-narcotic medications, physical or psychological therapies, surface electrical stimulation, and spinal cord stimulation, all of which have practical limitations that prevent widespread use.

Undergoing a surgical procedure and recovering from it is generally a painful process, emotionally and physically. There remains a need in the art of surgical preparation and/or pain management for improved systems and methods to be used to ready an animal body, especially, a human, for surgery and/or to assist in the recovery of the body after a surgical procedure. Such assistance in recovery of the body after a surgical procedure may include measures to alleviate post-operative pain and to prevent the onset or development of chronic post-operative pain. There is, therefore, a need for an improved pain treatment system and method for relief of post-operative pain, especially pain following amputation surgery.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

An embodiment contemplates a system to alleviate pain following surgical amputation or removal of an extremity, limb, body part, or body tissue including any combination of the following features:
a coiled wire lead having a thickness and a diameter that are appropriate for percutaneous insertion into a portion of a body that is proximal to or will become remain part of a residual limb or extremity following surgical amputation;
an electrode formed integrally on the lead, wherein the electrode is positioned: i) inside the body when the system is functioning, and ii) at a therapeutically effective distance from a portion of at least one nerve that will be transected or cut during the surgical amputation;
an electrical stimulation device operatively coupled to the lead to apply electrical stimulation through the at least one electrode to at least one nerve that will be transected or cut, to create comfortable sensations in a region of residual or phantom pain;
wherein the electrical stimulation also causes comfortable sensations within the body in an area outside of the region of residual or phantom pain;
wherein the electrical stimulation occurs proximal to the residual limb or extremity;
wherein the electrical stimulation includes a first parameter selected from a group consisting of: frequency, pulse duration, amplitude, duty cycle, pattern of stimulus pulses, polarity, a predetermined number of phases, and waveform shape;
wherein the electrical stimulation includes a second parameter selected from a group consisting of: frequency, pulse duration, amplitude, duty cycle, pattern of stimulus pulses, polarity, a predetermined number of phases, and waveform shape;
wherein the first and second parameters are identical and each has a defined value and wherein the defined values for the first and second parameters are different;
wherein the electrical stimulation does not damage the at least one nerve that will be transected or cut;
wherein the region of residual or phantom pain is at or distal to a location of the surgical amputation, said location selected from a group consisting of: above knee, at knee, below knee, at ankle, foot, toe, above shoulder, at shoulder, above elbow, at elbow, below elbow, at wrist, hand, finger, and breast;
wherein the region of phantom pain is associated with amputation of appendages or extremities;
wherein the at least one nerve is a peripheral nerve;
wherein the at least one nerve that will be transected or cut is selected from a group consisting of a femoral nerve, a sciatic nerve, lateral femoral cutaneous nerve, an obturator nerve, median nerve, radial nerve, ulnar nerve, axillary nerve, musculocutaneous nerve, brachial plexus, lumbar plexus, sacral plexus, intercostal nerve, ilioinguinal nerve, iliohypogastric nerve, and intercostobrachial nerve; and
wherein the at least one nerve selected from the group includes at least one distal branch of the selected nerve or nerves.

In another embodiment contemplates a system to alleviate pain after an amputation surgery including any combination of the following features:
at least one electrode placed within a therapeutically effective distance from a peripheral nerve innervating a targeted region of pain resulting from the amputation surgery, said electrode positioned outside of the targeted region of pain;
an electrical stimulation device to deliver electrical stimulation through the at least one electrode;
wherein the electrical stimulation activates the peripheral nerve to evoke a comfortable sensation in the expected targeted region of pain but without functional nerve stimulation at a motor point of the peripheral nerve;
wherein the electrical stimulation device applies electrical stimulation through the at least one electrode after the amputation surgery evoking a second comfortable sensation.
wherein the electrode is positioned prior to the amputation surgery and wherein electrical stimulation delivered to the peripheral nerve prior to the surgery does not damage the peripheral nerve;
wherein the electrical stimulation prior to surgery includes a first set of electrical stimulation parameters;
wherein the electrical stimulation after the amputation surgery includes a set of second electrical stimulation parameters and wherein at least one of the second electrical stimulation parameters is different from at least one of the first electrical stimulation parameters;
wherein the at least one of the second electrical stimulation parameters includes at least one of frequency, pulse duration, amplitude, duty cycle, pattern of stimulus pulses, polarity, a predetermined number of phases, and waveform shape;
wherein the at least one electrode is located in tissue that is proximal from the region of pain;
wherein the amputation surgery includes transtibial or transfemoral lower limb amputation;
wherein the peripheral nerve is selected from the femoral nerve, the sciatic nerve, and the obturator nerve;
wherein the electrical stimulation device comprises a pulse generator;
wherein the pulse generator delivers the electrical stimulation to the peripheral nerve by way of a percutaneous lead attached to the electrode;
wherein the peripheral nerve, inclusive of any distal branch thereof, is selected from a group consisting of a femoral nerve, a sciatic nerve, lateral femoral cutaneous nerve, an obturator nerve, median nerve, radial nerve, ulnar nerve, axillary nerve, musculocutaneous nerve, brachial plexus, lumbar plexus, sacral plexus, intercostal nerve, ilioinguinal nerve, iliohypogastric nerve, and intercostobrachial nerve; and
wherein the electrical stimulation will not block motor or sensory function of a limb.

Another embodiment contemplates a kit for treatment of pain from amputation surgery comprising any combination of the following features:
a needle insertable into an animal body tissue;
a percutaneous lead operatively inserted into the needle, wherein the needle and the percutaneous lead are inserted into an insertion point of the animal body, whereby the needle is removable from the animal body tissue and the percutaneous lead is retained within the animal body; the systems described in the first or second embodiments, as indicated above;
wherein the electrical stimulation causes an area of comfortable sensations and the area of comfortable sensations is compared with the region of expected pain from the amputation surgery;
a test needle;
wherein the needle is an introducer needle;
an anchor attached to the percutaneous lead, the anchor configured to operatively retain the percutaneous lead at the insertion point during withdrawal of the needle from the animal body; and
wherein the amputation surgery is selected from a group consisting of: limb amputation above knee, limb amputation at knee, limb amputation below knee, limb amputation at ankle, full or partial foot amputation, full or partial toe amputation, limb amputation above shoulder, limb amputation at shoulder, limb amputation above elbow, limb amputation at elbow, limb amputation below elbow, limb amputation at wrist, full or partial hand amputation, full or partial finger amputation, and mastectomy or breast amputation.

A still further embodiment contemplates a kit for treatment of pain from amputation surgery comprising any combination of the following features:
a needle insertable into an animal body tissue;
a percutaneous lead operatively inserted into the needle, wherein the needle and the percutaneous lead are inserted into an insertion point of the animal body, whereby the needle is removable from the animal body tissue and the percutaneous lead is retained within the animal body; and
the systems described in the first or second embodiments, as indicated above;
wherein the electrical stimulation causes an area of comfortable sensations and the area of comfortable sensations is compared with the region of expected pain from the amputation surgery;
a test needle;
wherein the needle is an introducer needle; and
an anchor attached to the percutaneous lead, the anchor configured to operatively retain the percutaneous lead at the insertion point during withdrawal of the needle from the animal body.

Another embodiment contemplates methods to alleviate pain following an amputation surgery based upon any combination of the following features:
inserting a coiled wire lead with at least one electrode percutaneously into a body within a therapeutically effective distance from at least one nerve to be transected;
wherein the at least one electrode is positioned outside of a region of pain expected from the amputation surgery;
applying electrical stimulation through the at least one electrode to the nerve to be transected, thereby activating nerve fibers that innervate the region of pain expected from the amputation surgery in advance of the amputation surgery and causing an area of comfortable sensations in the body, said area of comfortable sensations being compared with an area affected by the at least one nerve innervating the region of pain expected from the amputation surgery;
wherein electrical stimulation is provided to the nerve to be transected prior to the amputation surgery;
wherein the electrical stimulation is provided to the nerve to be transected after said nerve is transected during the amputation surgery;
inserting or instructing insertion of a coiled wire lead with at least one electrode percutaneously into a body within a therapeutically effective distance from at least one nerve to be transected;
wherein the at least one electrode is positioned outside of a region of pain expected from the amputation surgery; and
delivering or instructing delivery of electrical stimulation through the at least one electrode to the nerve to be transected, thereby activating nerve fibers that innervate the region of pain expected from the amputation surgery in advance of the amputation surgery and causing an area of comfortable sensations in the body, said area of comfortable sensations being compared with an area affected by the at least one nerve innervating the region of pain expected from the amputation surgery.

Other features and advantages of the present teachings are set forth in the following specification and attached drawings. The following description and the drawings disclose various illustrative aspects. Some improvements and novel aspects may be expressly identified, while others may be apparent from the description and drawings.

### DESCRIPTION OF THE DRAWINGS

The accompanying drawings illustrate various systems, apparatuses, devices and methods, in which like reference characters refer to like parts throughout, and in which:

FIGS. 1A and 1B are schematic anatomic views, respectively anterior and lateral, of a human peripheral nervous system.

FIG. 2 is a schematic anatomic view of a human spine, showing the various regions and the vertebrae comprising the regions.

FIGS. 3A and 3B are anatomic views of the spinal nerves of the lumbar plexus and the sacral plexus, and the brachial plexus.

FIGS. 4A and 4B are anatomic views of the femoral and sciatic nerves, respectively, in the lower limb.

FIG. 5 are anatomic view of the femoral nerve (FN) and sciatic nerve (SN) innervation of the leg, from the front and back, separately isolated to highlight skin, muscle, and bone, respective shown as (i), (ii), and (iii).

FIGS. 6A to 6D are views showing a percutaneous lead that can form a part of a peripheral nerve stimulation system, with the lead inside an introducer needle (6A and 6B) and placed remote to a nerve after the removal of the introducer needle (6C). After placement of the lead and removal of the introducer (6C), the lead may be connected to an external stimulator with a surface electrode as a return electrode, and an appropriate bandage placed over the site where the lead exits the skin (6D). In all views, dermis 103, tissue 104, and nerve 105 are shown separately.

FIG. 7 is a view of a package containing a peripheral nerve stimulation system and instructions for use.

FIGS. 8A and 8B are representative leads that can form a part of a peripheral nerve stimulation system.

FIGS. 9A and 9B are schematic anatomic views of a system for applying peripheral nerve stimulation to a femoral nerve.

FIGS. 10A and 10B are schematic anatomic views of a system for applying peripheral nerve stimulation to a sciatic nerve.

FIGS. 11A and 11B are schematic sectional anatomic views of systems for applying peripheral nerve stimulation to a femoral nerve and a sciatic nerve, where the trajectory areas for femoral nerve stimulation (FNS) and sciatic nerve stimulation (SNS) are defined by dashed lines. The needle and/or lead may directly approach a targeted nerve to within electrical proximity (11A) or may pass near/alongside but still within electrical proximity of a targeted nerve (11B).

FIG. 12A is a frontal and rear view showing the peripheral nerve stimulation system and one potential location of a surgical amputation. FIG. 12B shows the peripheral nerve stimulation system and one potential location (SS) of surgical removal of body tissue.

FIGS. 13A and 13B are idealized, diagrammatic views showing peripheral nerve stimulation systems. FIG. 13A shows the shoulder (axillary, suprascapular or lateral pectoral nerves) and arms/hand (radial, musculocutaneous, or ulnar nerves) areas, along with the target stimulation site (TSS), level of amputation (LoA), and potential areas of pain (PP). Similarly, FIG. 13B shows the Femoral or sciatic nerves, with the same indications. In both cases, afferent signals block pain transmission to the brain (B).

FIGS. 14A, 14B, and 14C are, respectively speaking, front and rear views of the areas of post-amputation pain (PP) in FIG. 14A, stimulation-evoked comfortable sensations (CS) that overlap the areas of post-amputation pain in FIG. 14B, and stimulation-evoked comfortable sensations that cover a region that includes within it the areas of post-amputation pain in FIG. 14C on a diagram of the body following transfemoral amputation, relative to the level of amputation (LoA).

FIGS. 15A and 15B are, respectively speaking, front and rear views of the areas of post-amputation pain (PP) in FIG. 15A, and areas of pain relief (PR) during stimulation that overlap the areas of post-amputation pain in FIG. 15B on a diagram of the body following transfemoral amputation, relative to the level of amputation (LoA).

FIGS. 16A, 16B, and 16C are, respectively speaking, front views of areas of pain following surgical removal of body tissue (PS) in FIG. 16A, stimulation-evoked comfortable sensations (CS) that overlap the areas of post-surgical removal pain in FIG. 16B, and areas of pain relief (PR) during stimulation that overlap the areas of post-surgical removal pain in FIG. 16C on a diagram of the body, relative to the location of surgical removal of the body tissue (SS).

### DETAILED DESCRIPTION

Reference will now be made to exemplary embodiments, examples of which are illustrated in the accompanying drawings. It is to be understood that other embodiments may be utilized and structural and functional changes may be made. Moreover, features of the various embodiments may be combined or altered. As such, the following description is presented by way of illustration only and should not limit in any way the various alternatives and modifications that may be made to the illustrated embodiments. In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

Moreover, the terms “patient,” “animal” “animal body” and the like are employed interchangeably throughout the subject specification, unless context suggests otherwise or warrants a particular distinction among the terms. Still further, the terms “user,” “clinician,” “medical professional,” “doctor,” “surgeon,” and the like are employed interchangeably through the subject specification, unless context suggests otherwise or warrants a particular distinction among the terms.

“Logic” refers to any information and/or data that may be applied to direct the operation of a processor. Logic may be formed from instruction signals stored in a memory (e.g., a non-transitory memory). Software is one example of logic. In another aspect, logic may include hardware, alone or in combination with software. For instance, logic may include digital and/or analog hardware circuits, such as hardware circuits comprising logical gates (e.g., AND, OR, XOR, NAND, NOR, and other logical operations). Furthermore, logic may be programmed and/or include aspects of various devices and is not limited to a single device.

Any elements described herein as singular can be pluralized (i.e., anything described as “one” can be more than one) unless otherwise noted. Any species element of a genus element can have the characteristics or elements of any other species element of that genus. The described configurations, elements or complete assemblies and methods and their elements for carrying out the present teachings, and variations of aspects of the present teachings can be combined and modified with each other in any combination.

The present disclosure relates to systems and methods for placing one or more leads in tissues for providing electrical stimulation to the tissue to treat pain by providing an electrical stimulation device having at least one percutaneous lead adapted for insertion within tissue of an animal body and a pulse generator operatively coupled with the at least one lead, wherein the pulse generator is configured to stimulate at least one nerve innervating a region of pain following amputation surgery and/or surgery for removal of body tissue or portion thereof. The system and method may also generate comfortable sensations and/or pain relief in the region of the surgery, including sensations and/or relief perceived to be in the body tissues that were actually amputated/removed during that surgery.

A kit for treatment of pain following amputation surgery and/or surgery for removal of body tissue or a portion thereof is also contemplated. The kit may include a needle insertable into an animal body tissue, at least one percutaneous electrode lead operatively inserted into the needle, wherein the needle and at least one percutaneous lead are inserted into an insertion point of the animal body. The needle can be removed from the animal body tissue and the at least one percutaneous electrode lead may be retained within the animal body. A pulse generator is operatively coupled with the at least one electrode lead, wherein the pulse generator is configured to stimulate at least one nerve innervating a region of pain following an amputation surgery and/or surgery for removal of body tissue or a portion thereof.

Methods to alleviate pain following an amputation surgery and/or surgery for removal of body tissue or a portion thereof are also disclosed. One such method may include inserting at least one electrode/lead within a therapeutically effective distance from at least one nerve and applying electrical stimulation through the at least one electrode to affect the at least one nerve innervating a region of pain following the amputation surgery. In this method, the electrical stimulation does not cause pain.

The systems, methods, kits, and associated devices—as well as instructions for use of the same—may prevent the development of chronic post-amputation pain, for example phantom limb pain or residual limb pain. In each instance, at least one electrode may be inserted in a patient within a therapeutically effective distance from at least one nerve. Electrical stimulation is then applied through the at least one electrode without causing pain or impairing normal bodily functions. This stimulation affects the at least one nerve innervating a region of pain following the amputation surgery or a region that may develop multiple types of pain, such as but not limited to acute, post-surgical, sub-acute, transitional, and/or chronic pain following the amputation surgery.

I. The Peripheral Nervous System—Anatomic Overview

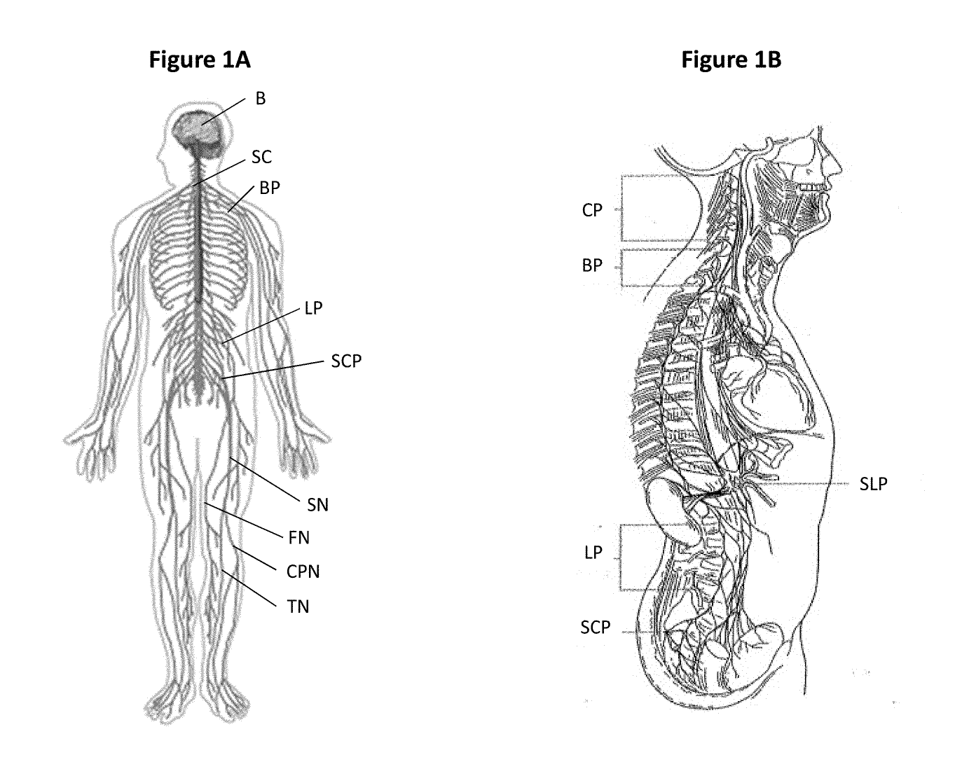

As generally shown in FIGS. 1A and 1B, the peripheral nervous system consists of nerve fibers and cell bodies outside the central nervous system (i.e., the brain and the spinal column) that conduct impulses to or away from the central nervous system. The peripheral nervous system is made up of nerves (called spinal nerves) that connect the central nervous system with peripheral structures. The spinal nerves of the peripheral nervous system arise from the spinal column and exit through intervertebral foramina in the vertebral column (spine). The afferent, or sensory, fibers of the peripheral nervous system convey neural impulses to the central nervous system from the sense organs (e.g., the eyes) and from sensory receptors in various parts of the body (e.g., the skin, muscles, etc.). The efferent, or motor, fibers convey neural impulses from the central nervous system to the effector organs (muscles and glands).

The somatic nervous system (SNS) is the part of the peripheral nervous system associated with the voluntary control of body movements through the action of skeletal muscles, and with reception of external stimuli, which helps keep the body in touch with its surroundings (e.g., touch, hearing, and sight). The system includes all the neurons connected with skeletal muscles, skin and sense organs. The somatic nervous system consists of efferent nerves responsible for sending central nervous signals for muscle contraction. A somatic nerve is a nerve of the somatic nervous system.

A. Spinal Nerves

A typical spinal nerve arises from the spinal cord by rootlets that converge to form two nerve roots, the dorsal (sensory) root and the ventral (motor) root. The dorsal and ventral roots unite into a mixed nerve trunk that divides into a smaller dorsal (posterior) primary ramus and a much larger ventral (anterior) primary ramus. The posterior primary rami serve a column of muscles on either side of the vertebral column, and a narrow strip of overlying skin. All of the other muscle and skin is supplied by the anterior primary rami.

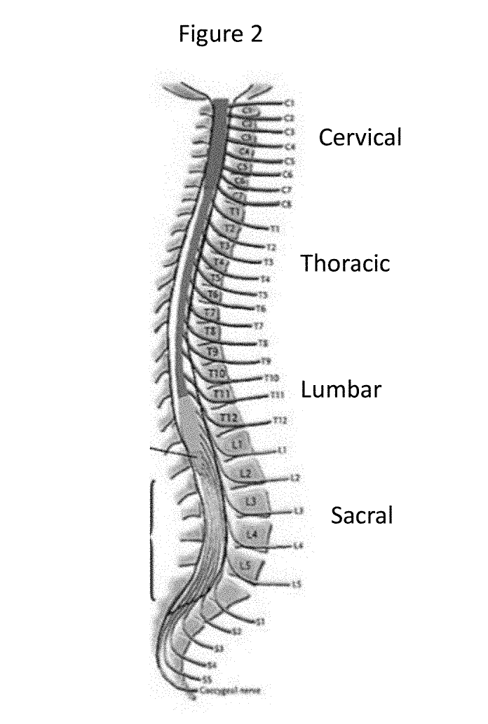

The nerve roots that supply or turn into peripheral nerves can be generally categorized by the location on the spine where the roots exit the spinal cord, i.e., as generally shown in FIG. 2, cervical (generally in the head/neck, designated C1 to C8), thoracic (generally in chest/upper back, designated T1 to T12), lumbar (generally in lower back, designated L1 to L5); and sacral (generally in the pelvis, designated S1 to S5). All peripheral nerves can be traced back (proximally toward the spinal column) to one or more of the spinal nerve roots in either the cervical, thoracic, lumbar, or sacral regions of the spine. The neural impulses comprising pain felt in a given muscle or cutaneous region of the body pass through spinal nerves and (usually) one or more nerve plexuses. The spinal nerves begin as roots at the spine, and can form trunks that divide by divisions or cords into branches that innervate skin and muscles.

B. Nerves of the Sacral Plexus

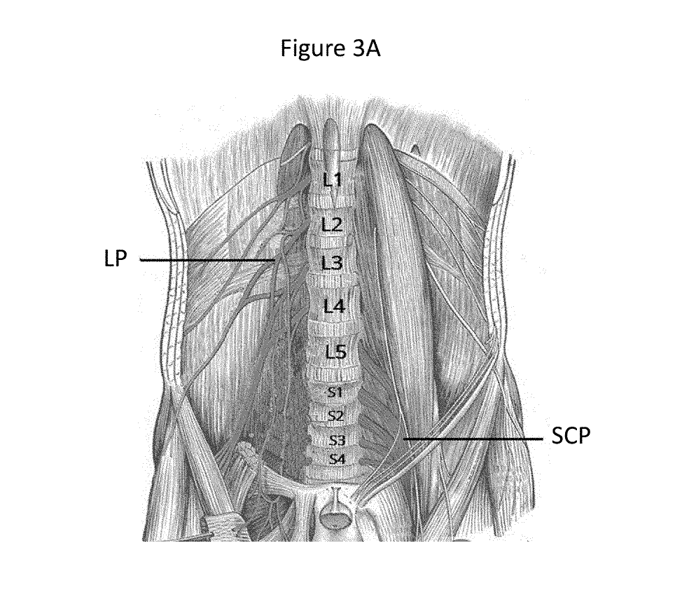

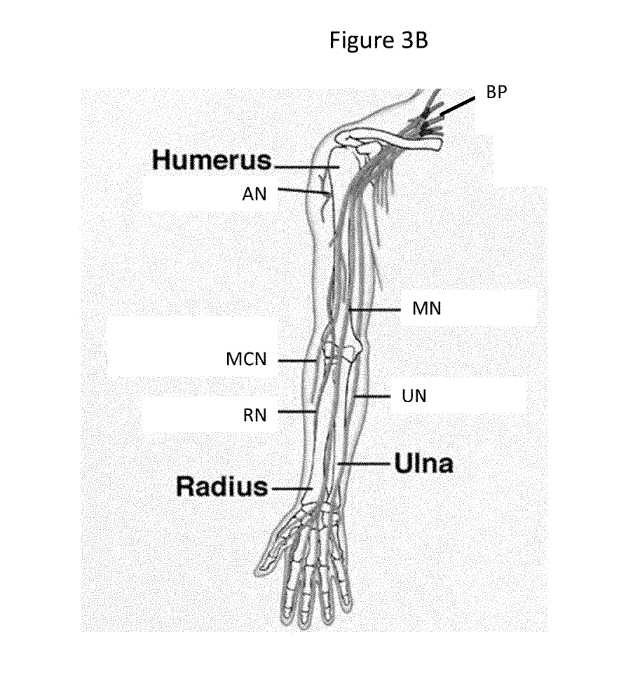

The sacral plexus SCP (FIG. 3A) provides motor and sensory nerves for the posterior thigh, most of the lower leg, and the entire foot.

1. The Sciatic Nerve

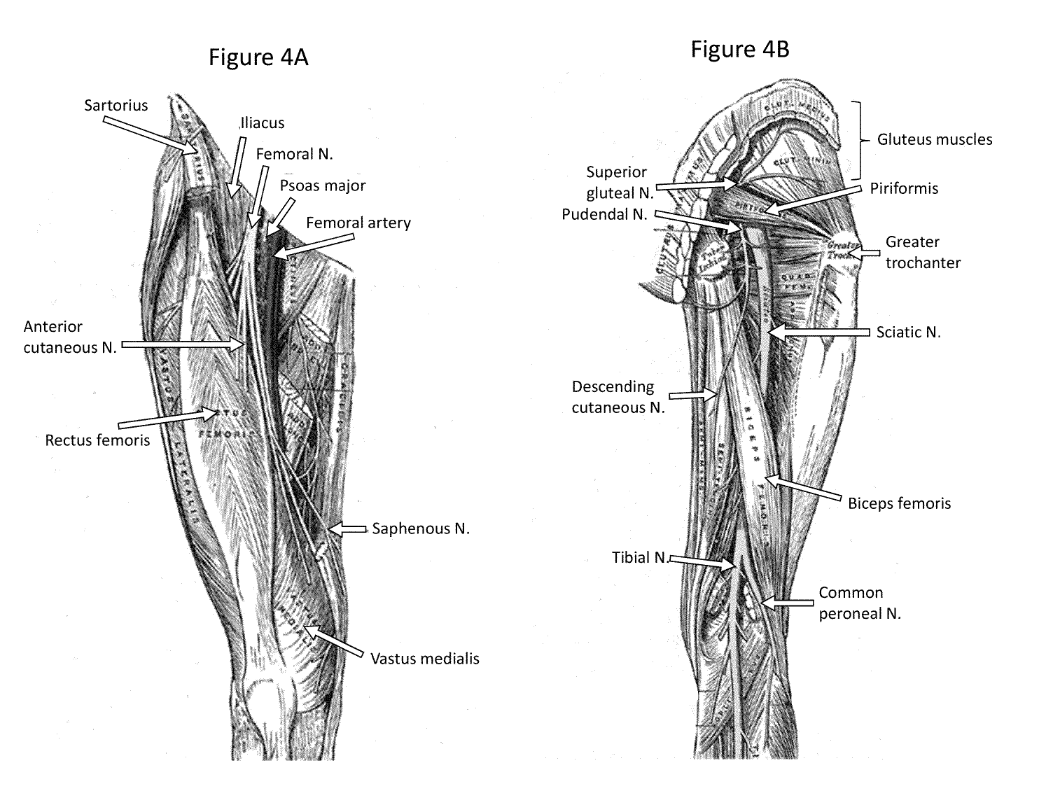

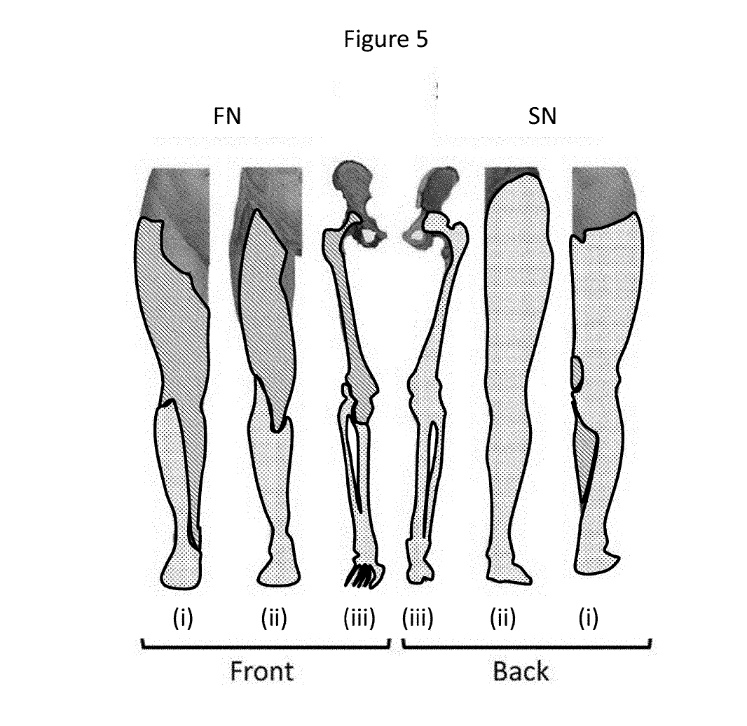

As shown in FIGS. 1A and 4B, the sciatic nerve (also known as the ischiatic nerve) arises from the sacral plexus. It begins in the lower back and runs through the buttock and down the lower limb. The sciatic nerve supplies nearly the whole of the skin of the leg, the muscles of the back of the thigh, and those of the leg and foot. It is derived from spinal nerves L4 through S3. It contains fibers from both the anterior and posterior divisions of the lumbosacral plexus.

The nerve gives off articular and muscular branches. The articular branches (rami articulares) arise from the upper part of the nerve and supply the hip-joint, perforating the posterior part of its capsule; they are sometimes derived from the sacral plexus. The muscular branches (rami musculares) innervate the following muscles of the lower limb: biceps femoris, semitendinosus, semimembranosus, and adductor magnus. The nerve to the short head of the biceps femoris comes from the common peroneal part of the sciatic, while the other muscular branches arise from the tibial portion, as may be seen in those cases where there is a high division of the sciatic nerve.

The muscular branch of the sciatic nerve SN eventually gives off the tibial nerve TN and common peroneal nerve CPN (all shown in FIG. 1A), which innervates the muscles of the (lower) leg. The tibial nerve TN innervates the gastrocnemius, popliteus, soleus and plantaris muscles and the knee joint. It also goes on to innervate all muscles of the foot except the extensor digitorum brevis (which is innervated by the peroneal nerve). Additionally, the relative locations of the femoral nerve FN, sacral plexus SCP, cervical plexus CP, solar plexus SLP, lumbar plexus LP, brachial plexus BP, spinal cord SC, and brain B are also shown in FIGS. 1A and 1B, while the pudendal, coccygeal, and sacral nerves can be located in FIGS. 3A and/or 4B.

C. Nerves of the Lumbar Plexus

The lumbar plexus (see FIG. 3A) provides motor, sensory, and autonomic fibers to gluteal and inguinal regions and to the lower extremities. The gluteal muscles are the three muscles that make up the buttocks: the gluteus maximus muscle, gluteus medius muscle and gluteus minimus muscle. The inguinal region is situated in the groin or in either of the lowest lateral regions of the abdomen.

1. The Iliohypogastric Nerve

The iliohypogastric nerve (see FIG. 3A) runs anterior to the psoas major on its proximal lateral border to run laterally and obliquely on the anterior side of quadratus lumborum. Lateral to this muscle, it pierces the transversus abdominis to run above the iliac crest between that muscle and abdominal internal oblique. It gives off several motor branches to these muscles and a sensory branch to the skin of the lateral hip. Its terminal branch then runs parallel to the inguinal ligament to exit the aponeurosis of the abdominal external oblique above the external inguinal ring where it supplies the skin above the inguinal ligament (i.e. the hypogastric region) with the anterior cutaneous branch.

2. The Ilioinguinal Nerve

The ilioinguinal nerve (see FIG. 3A) closely follows the iliohypogastric nerve on the quadratus lumborum, but then passes below it to run at the level of the iliac crest. It pierces the lateral abdominal wall and runs medially at the level of the inguinal ligament where it supplies motor branches to both transversus abdominis and sensory branches through the external inguinal ring to the skin over the pubic symphysis and the lateral aspect of the labia majora or scrotum.

3. The Lateral Cutaneous Femoral Nerve

The lateral cutaneous femoral nerve (see FIG. 3A) pierces psoas major on its lateral side and runs obliquely downward below the iliac fascia. Medial to the anterior superior iliac spine it leaves the pelvic area through the lateral muscular lacuna. In the thigh, it briefly passes under the fascia lata before it breaches the fascia and supplies the skin of the anterior thigh.

4. The Obturator Nerve

The obturator nerve (see FIG. 3A) leaves the lumbar plexus and descends behind psoas major on its medial side, then follows the linea terminalis and exits through the obturator canal. In the thigh, it sends motor branches to obturator externus before dividing into an anterior and a posterior branch, both of which continue distally. These branches are separated by adductor brevis and supply all thigh adductors with motor innervation: pectineus, adductor longus, adductor brevis, adductor magnus, adductor minimus, and gracilis. The anterior branch contributes a terminal, sensory branch that passes along the anterior border of gracilis and supplies the skin on the medial, distal part of the thigh.

5. The Femoral Nerve

The femoral nerve (see FIGS. 1A, 3A, and FIG. 10A) is the largest and longest nerve of the lumbar plexus. It gives motor innervation to iliopsoas, pectineus, sartorius, and quadriceps femoris; and sensory innervation to the anterior thigh, posterior lower leg, and hind foot. It runs in a groove between psoas major and iliacus giving off branches to both muscles. In the thigh, it divides into numerous sensory and muscular branches and the saphenous nerve, its long sensory terminal branch which continues down to the foot.

The femoral nerve has anterior branches (intermediate cutaneous nerve and medial cutaneous nerve) and posterior branches. The saphenous nerve (branch of the femoral nerve) provides cutaneous (skin) sensation in the medial leg. Other branches of the femoral nerve innervate structures (such as muscles, joints, and other tissues) in the thigh and around the hip and knee joints. As an example, branches of the femoral nerve innervate the hip joint, knee joint, and the four parts of the Quadriceps femoris (muscle): Rectus femoris (in the middle of the thigh) originates on the ilium and covers most of the other three quadriceps muscles. Under (or deep to) the rectus femoris are the other three of the quadriceps muscles, which originate from the body of the femur. Vastus lateralis (on the outer side of the thigh) is on the lateral side of the femur. Vastus medialis (on the inner part thigh) is on the medial side of the femur. Vastus intermedius (on the top or front of the thigh) lies between vastus lateralis and vastus medialis on the front of the femur. Branches of the femoral nerve often innervate the pectineus and sartorius muscles.

D. Nerves of the Brachial Plexus

1. Median Nerve

The median nerve MN originates from the lateral and medial cords of the brachial plexus BP (FIG. 3B). The nerve enters the arm from the axilla (below the shoulder) and runs alongside the brachial artery between the biceps brachii and brachialis muscles. The median nerve MN gives off an articular branch in the upper arm that supplies the elbow joint. As it courses through the forearm, the anterior interosseous branch and palmar cutaneous branch split off, then the median nerve MN itself enters the hand through the carpal tunnel, where it branches several times to supply sensory and motor innervation of various compartments of the hand and fingers. The median nerve MN innervates the flexors in the forearm, with the exception of two flexors that supply the fourth and fifth digits, and supplies motor innervation and cutaneous sensory innervation of much of the hand.

2. Radial Nerve

The median nerve MN supplies the anterior portion of the arm and the radial nerve RN supplies the posterior portion of the limb. It originates from the posterior cord of the brachial plexus (FIG. 3B), dividing ultimately into a deep branch and a superficial branch. The nerve supplies branches that innervate the medial and lateral heads of the triceps brachii, and then courses down the arm before piercing the lateral intermuscular septum and entering the anterior compartment of the arm. In the forearm, the superficial and deep branches of the radial nerve serve primarily sensory and motor roles, respectively. The superficial, sensory branch supplies parts of the thumb, index, middle, and part of the ring fingers. The deep branch supplies the supinator muscle and other forearm muscles, mostly including extensor muscles.

3. Ulnar Nerve

The ulnar nerve UN originates from part of the medial cord of the brachial plexus and descends on the posteromedial side of the humerus (FIG. 3B). It is exposed for several centimeters as it passes behind the medial epicondyle in the cubital tunnel at the elbow, and is commonly injured, entrapped, or bumped at that site, creating the tingling sensation commonly known as “hitting the funny bone.” The ulnar nerve supplies the flexor carpi ulnaris and part of the flexor digitorum profundus in the forearm, and travels down the ulna to enter the palm of the hand through Guyon's canal. The ulnar nerve has sensory function, supplying the fifth digit and medial fourth digit and palm. It also has motor function, innervating muscles in the forearm and the hand via its muscular, deep, and superficial branches.

4. Axillary Nerve

The axillary nerve AN originates from the posterior cord of the brachial plexus below the shoulder in the axilla (or armpit) (FIG. 3B). It passes downward anterior to the subscapularis muscle, and then moves posteriorly between teres major and teres minor, lateral to the triceps brachii and medial to the neck of the humerus. The nerve branches then into an anterior branch, posterior branch, and a motor branch that supplies the triceps brachii. The anterior branch innervates the deltoid muscle and provides cutaneous branches, and the posterior branch supplies teres minor and the posterior deltoid. The nerve has mixed function, with motor innervation of the deltoid, teres minor, and triceps brachii, and sensory innervation of the shoulder joint and overlying skin, especially over the inferior portion of the deltoid.

5. Musculocutaneous Nerve

The musculocutaneous nerve MCN is derived from the lateral cord of the brachial plexus. The nerve typically courses between the biceps brachii and brachialis muscles (FIG. 3B), innervating both muscles along with the coracobrachialis muscle. The actual course of this nerve relative to the coracobrachialis, brachialis, and biceps brachii may vary in some individuals. A small branch may innervate the pronator teres, and the nerve may supply sensory innervation of the dorsal surface of the thumb in some individuals with aberrations in the radial nerve (which typically innervates the dorsal surface of the thumb).

E. Trunk Nerves

1. Intercostal Nerve

The intercostal nerves arise from the thoracic spinal nerves from T1 to T11. The intercostal nerves generally innervate the walls of the thorax and the thoracic pleura, and nerves arising from lower thoracic levels (level 7-11) also supply the abdominal wall and abdominal peritoneum. The exact function, location and course, and structures innervated vary depending on the thoracic level of the nerve, but the intercostal nerves are mixed nerves, with both motor and sensory function.

2. Intercostobrachial Nerve

The intercostobrachial nerves are cutaneous branches of the intercostal nerves. Two levels in particular are likely to have intercostobrachial nerves. The second intercostobrachial nerve arises from the lateral cutaneous branch of the second intercostal nerve, crossing the axilla and supplying skin on the upper half of the medial and posterior arm. This nerve is often the source of the referred cardiac pain. The third intercostobrachial nerve arises from the lateral cutaneous branch of the third intercostal nerve, supplying filaments to the axilla and medial side of the arm.

II. The System

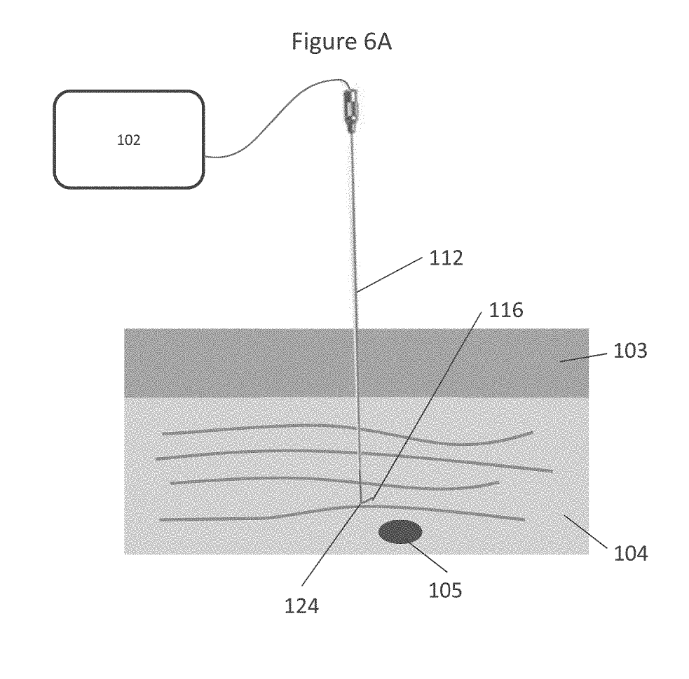

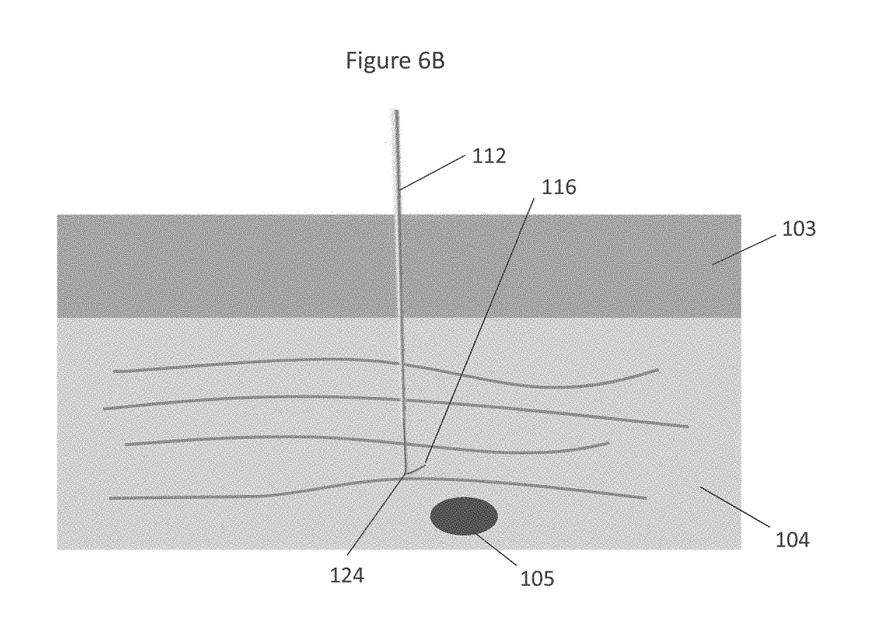

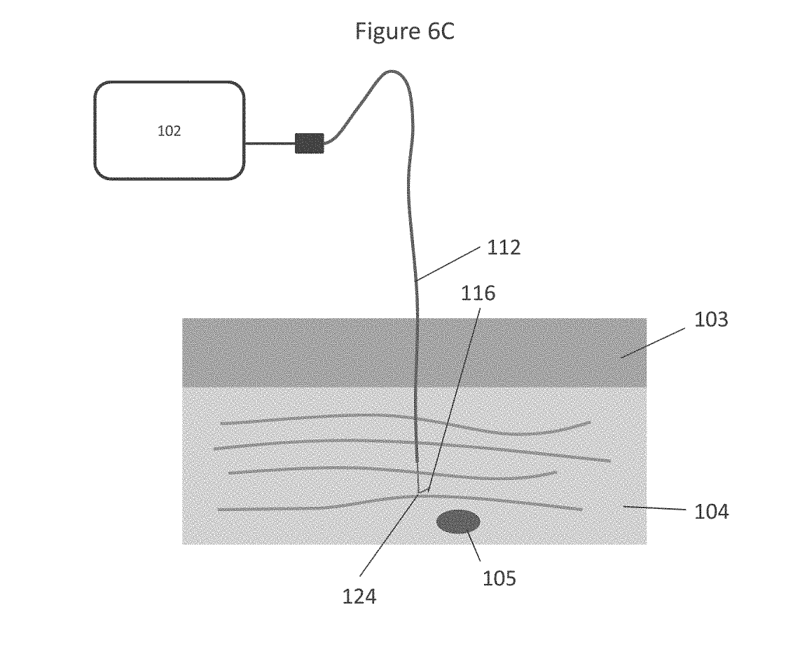

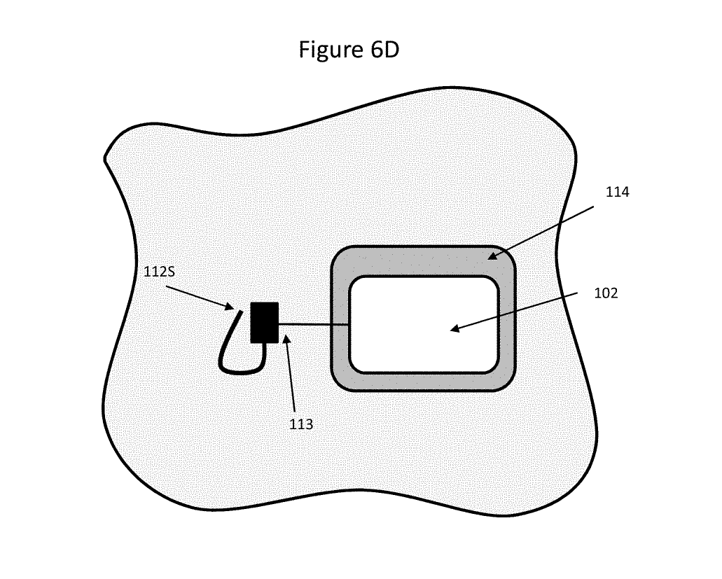

Shown in FIGS. 6A, 6B, 6C and 6D is an electrical stimulation device 102 configured to treat post-operative pain, especially pain following amputation surgery. Here, an amputation surgery may include amputation at any level of the upper and lower extremities and/or surgery related to the removal of body tissue. Non-limiting examples include above knee, at knee, below knee, at ankle, full or partial foot, full or partial toe, above shoulder, at shoulder, above elbow, at elbow, below elbow, at wrist, full or partial hand, full or partial finger amputation, mastectomy or breast amputation, and removal of other appendages, extremities, or body parts. Further, amputation surgery may include removal of organs or other bodily tissue.

Amputation is the removal of part of the body, and is commonly perceived as relating to amputation of extremities, limbs, or portions of extremities or limbs. However, it is to be appreciated that other parts of the body (e.g., nerve, muscle tissue, adipose tissue, connective tissue, bone, ligaments, tendons, joints, organs, masses (including both cancerous masses and non-cancerous masses), and other structures or tissues in the body) can be removed and/or amputated in whole and/or in part and are included as part of this disclosure. The removal or amputation of any body part(s) or tissue(s) can lead to significant pain, disability, dysfunction, or loss of function. The pain caused by, anticipated or expected from, and/or following the removal or amputation of the body part(s) or tissue(s), which may include an extremity, limb, appendage, part of an extremity, limb, or appendage, and/or a part of the body which is not an extremity, limb, appendage, or part of an extremity, limb, or appendage and is inside the body in whole or in part (such as a nerve, muscle tissue, adipose tissue, connective tissue, bone, ligaments, tendons, joints, organs, masses (including both cancerous masses and non-cancerous masses), and other structures or tissues in the body) may be treated and reduced or relieved by the present invention. The reduction and/or relief of pain provided by the present disclosure following amputation or removal of the body part or tissue may then lead to reduction of interference of pain in daily or usual activities, reduction of disability, improvement in function (without functional nerve stimulation at a motor point), and/or improvement in quality of life.

A. Stimulation of Peripheral Nerves

The peripheral nerve system, device, method, and instructions for use of systems, devices, and/or methods incorporate features of the present teachings. The system, device, method, and instructions for use of systems, devices, and/or methods may identify a region where there is a local manifestation of pain. The system, device, method, and instructions for use of systems, devices, and/or methods may also identify a region where there may be a manifestation of chronic pain at a later time, for example after an amputation surgery. The region of pain may comprise any appropriate portion of the body, e.g., tissue, skin, bone, a joint, muscle, or an appropriate portion of the body that was removed during amputation surgery or other surgical removal of tissue.

The system, device, method, and instructions for use of systems, devices, and/or methods may identify one or more spinal nerves located distant (e.g., any combination of one tenth increments between 0.5 cm and 3.0 cm) from the region where pain is manifested, through which neural impulses comprising the pain pass. A given spinal nerve that is identified may comprise a nerve trunk located in a nerve plexus, or a division and/or a cord of a nerve trunk, or a nerve branch, or a nerve plexus provided that it is upstream or cranial of where the nerve innervates the region affected by the pain. Table 1 provides non-limiting examples of regions of pain that may be innervated by specific nerves.

The given spinal nerve may be identified by medical professionals, doctor, surgeon or clinician using textbooks of human anatomy along with their knowledge of the site and the nature of the pain or injury, as well as by physical manipulation and/or imaging, e.g., by ultrasound, fluoroscopy, or X-ray examination, of the region where pain is manifested. A desired criterion of the selection may include identifying the location of tissue in a therapeutically effective distance (e.g., any combination of one tenth increments between 0.5 cm and 3.0 cm) from the nerve or passage, which tissue may be accessed by placement of one or more stimulation electrodes 116, aided if necessary by ultrasonic or electro-location techniques. A therapeutically effective distance may be defined to mean the placement of a lead adjacent to a nerve, preferably at a distance remote or removed from the nerve (e.g., between 0.1 cm and 4.0 cm, between 0.2 cm and 3.5 cm, between 0.5 cm and 3.0 cm, and/or greater than 0.1 cm or 0.2 cm) so as to allow preferential activation of targeted fibers without activation of non-target fibers. The nerve identified may comprise a targeted peripheral nerve. The tissue identified may comprise the “targeted tissue.” See Table 1 for further details.

TABLE 1
Examples of peripheral nerves and the regions the nerves innervate that can become the
location of pain following an amputation surgery

| | Region of Innervation | |
|---|---|---|
| Nerve | (Potential Region of Pain) | Example Amputation Surgery |
| Sciatic nerve | Posterior lower limb | Transtibial or transfemoral |
| | | amputation |
| Iliohypogastric nerve | Lateral hip and lower abdomen | Abdominal tissue removal |
| Ilioinguinal nerve | Lower abdomen and genitalia | Abdominal or genital tissue |
| | | removal |
| Lateral femoral cutaneous nerve | Anterolateral thigh | Transfemoral amputation |
| Obturator nerve | Distal medial thigh | Transfemoral amputation |
| Femoral nerve | Anterior lower limb | Transtibial or transfemoral |
| | | amputation |
| Median nerve | Forearm and digits | Hand or lower arm amputation |
| Ulnar nerve | Palm and digits | Hand or lower arm amputation |
| Radial nerve | Hand and digits | Hand or lower arm amputation |
| Musculocutaneous nerve | Upper limb | Upper arm amputation |
| Axillary nerve | Shoulder and upper limb | Upper arm amputation or |
| | | shoulder disarticulation |
| Intercostal nerve | Abdominal wall | Mastectomy or thoracic tissue |
| | | removal |
| Intercostobrachial nerve | Proximal medial upper limb | Upper arm amputation |

The electrical stimulation device 102 may include one or more leads 112 having one or more electrodes 116 adapted for insertion into in any tissue of the body in electrical proximity but away from nerves. In a preferred embodiment, the electrical stimulation device 102 may include a single percutaneous lead 112 with a single electrode 116 on the percutaneous lead 112 such that the location of the percutaneous lead 112 determines the location of the one electrode 116 relative to the target nerve. This location of lead(s) 112 may improve recruitment of targeted nerves for therapeutic purposes, such as for the treatment of pain. In such embodiments, the electrode 116 may be integrally formed with the lead 112 or may be monolithically formed with the lead 112.

The electrodes 116 of the electrical stimulation device 102 may be percutaneously inserted using the percutaneous lead(s) 112. The system and method may place the one or more leads 112 with its electrode 116 in the targeted tissue in electrical proximity to but spaced away from the targeted peripheral nerve. The system, device, or method may apply electrical stimulation through the one or more stimulation electrodes 116 to electrically activate or recruit the targeted peripheral nerve that conveys the neural impulses comprising the pain to the spinal column.

Electrical stimulation of the peripheral nerve may generate comfortable sensations, or paresthesia, in the region of innervation of the nerve, which may include body tissue (limb, extremity, portion of a limb or extremity, or other type of tissue) that was surgically removed or amputated. Stimulation may generate a perception of sensation from tissue that is no longer present after surgical removal, and the activation of the peripheral nerve to generate comfortable sensations may offset painful signals caused by the amputation or surgery. Activation of the peripheral nerve to generate comfortable sensations may reduce pain in acute or subacute setting after amputation or surgery to remove tissue, and/or may prevent the development of chronic pain in the phantom (e.g., the tissue that was surgically removed) or the remaining tissue in the innervation region of the targeted peripheral nerve. Stimulation may also be delivered so as to provide pain relief without evoking comfortable sensations or paresthesia. Stimulation may be delivered using sub-threshold (i.e., below the threshold for sensation or perception) or other parameters that provide relief of pain without producing sensations or paresthesia.

The paresthesia and pain relief produced by this electrical stimulation is unexpected when compared to previously known electrical stimulation techniques. These previous schemes addressed scenarios where pain relief is either provided to existing body tissue and/or to body tissue proximate to replacement objects (e.g., an artificial joint). In the present teachings, tissue is gone and not replaced by natural or artificial structures, so there is an absence of normal/healthy inputs to the central nervous system leading to imbalance between noxious/non-noxious inputs. Stated differently, the system, device, and methods disclosed herein deliver “normal” or non-painful or non-noxious neural input to rebalance the signals from the periphery to the central nervous system to reduce pain and to prevent the development of chronic pain.

As used herein, “comfortable sensations” generally means that pain levels, as experienced by the patient, do not change or, more preferably, decrease over a given period of time. Thus, any standard metric for pain can be employed at selected intervals, during the treatment and/or over a period of treatments. The pain level trend over that period is indicative of comfortable sensations. Other non-quantitative feedback, again provided by a given patient, also may augment or provide a further definition of “comfortable sensations.” Still other indicators include reports of tingling, paresthesia, and the like. In some cases, it may even be possible to use medical instrumentation to detect and/or quantify responses within the patient's body that are suggestive of pain or a lack thereof.

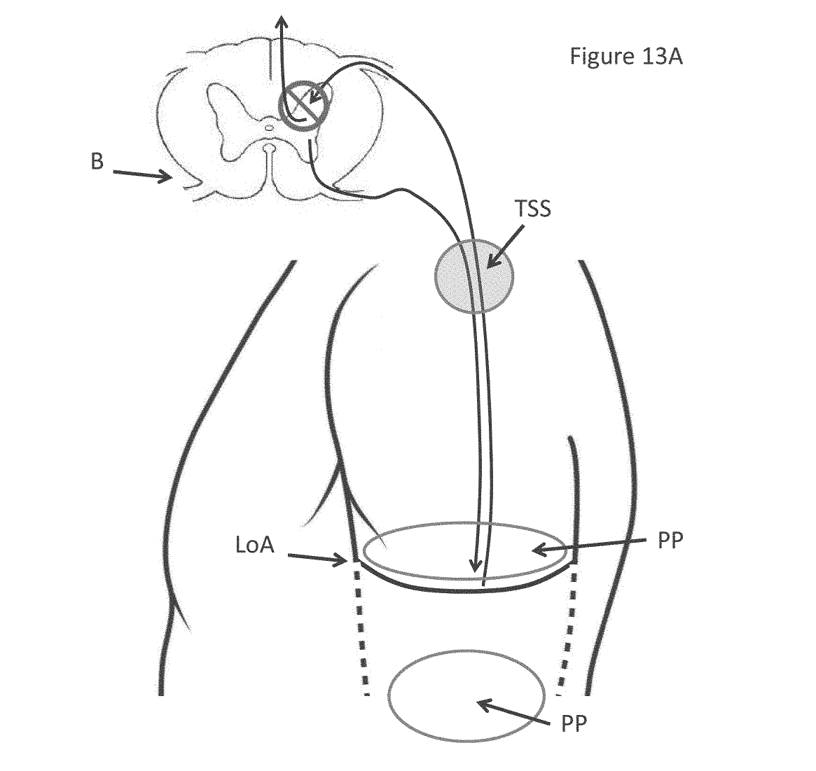

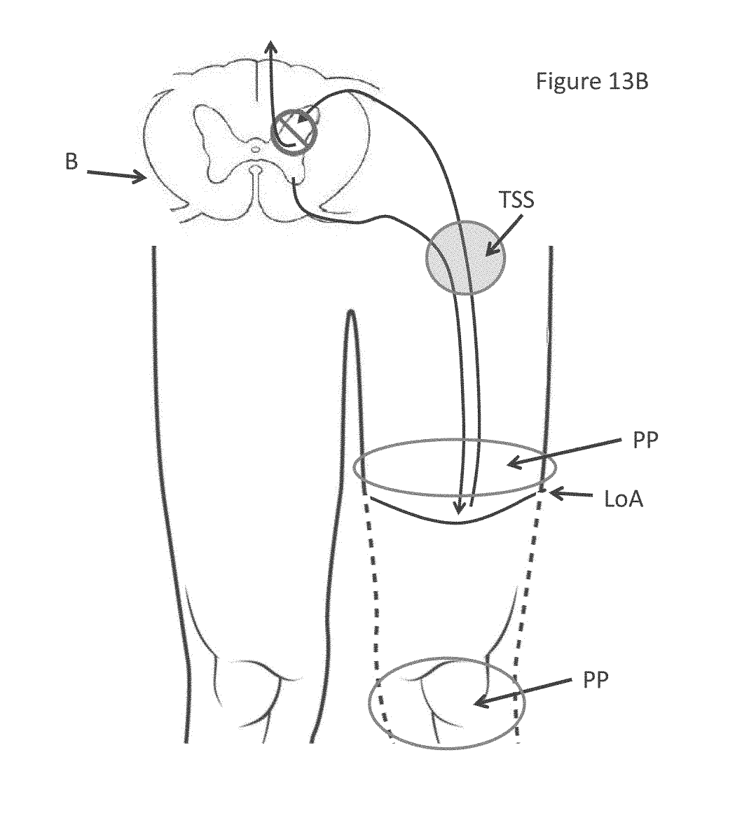

The system, device, method, and instructions for use of systems, devices, and/or methods may apply electrical stimulation to peripheral nerves throughout the body, including, without limitation, the body of a person. By way of a non-limiting example, the peripheral nerves may comprise one or more spinal nerves in the brachial plexus, to treat pain in the shoulders, arms, and hands after amputation (see FIG. 13A); and/or one or more spinal nerves in the lumbar plexus or sacral plexus, to treat pain in the thighs, knees, calves, and feet (FIG. 13B). The system, device, method, and instructions for use of systems, devices, and/or methods may also apply electrical stimulation to one or more spinal nerves in the cervical plexus, to treat pain in the shoulders.

The system, device, method, and instructions for use of systems, devices, and/or methods may reduce post-amputation pain by applying electrical stimulation to one or more peripheral nerves throughout the body before, during, and/or after the amputation. Therapy may be applied before, during, and/or after the resulting pain (i.e., post-amputation pain) develops, including in the acute, sub-acute, or chronic phases after amputation. The present system is designed to deliver therapy to relieve pain before, during, and/or after amputation, and may also be applied in a combination of those times, either continuously or intermittently. As a non-limiting example, the system, devices, methods, and instructions are designed to deliver therapeutic stimulation (e.g., electrical stimulation of the nerve of which a portion will be amputated) before amputation and after amputation. The therapeutic stimulation may be active prior to the amputation surgery, optionally turned off/deactivated during the surgery, and turned on/activated during and/or at some designated time following the surgery. Also, placement of electrical stimulation system may occur before or in the acute phase after amputation surgery (e.g., where tissue is being removed permanently).

The system, device, method, and instructions for use of systems, devices, and/or methods apply electrical stimulation to one or more peripheral nerves throughout the body to enable comfortable sensations to be evoked in the region that will be amputated in the distribution of the amputated nerve. Neural signals will be evoked by the therapeutic stimulation and sent to the central nervous system to decrease the perception of pain and maintain a balanced neural state. That is, the present system generates comfortable sensations to balance and/or override the uncomfortable and painful sensations that can accompany an amputation. By providing augmented neural input and/or drive and/or comfortable sensations from the periphery that signal to the central nervous system where noxious and/or painful and/or uncomfortable stimuli are processed, a reduction or elimination of pain can be achieved. If the augmented neural input is delivered while the amputation heals, the present system may prevent, reduce, and/or mitigate pain or the development of pain in the acute, sub-acute, or chronic phases after amputation. Thus, the present system enables therapeutic stimulation to be delivered to prevent, mitigate, or reduce the development of chronic pain.

The therapeutic stimulation may be delivered with a temporary or permanent fully implantable system, which could include a fully implantable lead and an implantable pulse generator (IPG) which may be controlled by external devices (such as a patient and/or clinician programmer and/or controller). The IPG may be powered by a(n) internal source(s), such as a rechargeable battery, a primary cell or non-rechargeable battery, or other means. The implantable system may also be powered by external sources, including an external pulse transmitter, or other means. The therapeutic stimulation may also be delivered with a temporary or permanent external (e.g., not implanted) system, which could include a fully implantable or percutaneously implanted lead and an external pulse generator (EPG) which may be controlled directly by controls on the EPG or by external devices (such as a patient and/or clinician programmer and/or controller). The EPG may be powered by a(n) internal source(s), such as a rechargeable battery, a primary cell or non-rechargeable battery, or other means.

For example, if the big toe (or great toe) is the location of pain in the residual limb (i.e., the stump) and/or the phantom limb (i.e., the amputated toe) following an amputation surgery, the system, device, and method may identify and stimulate the deep branch of the common peroneal nerve at a location upstream or cranial of where the nerve innervates the muscle or skin of the pinky finger, e.g., in the ankle and/or calf, or may identify and stimulate the sciatic nerve or another nerve trunk even further upstream of the deep peroneal nerve at the knee, thigh, or upper leg. If electrical stimulation activates the target peripheral nerve sufficiently at the correct intensity, then the patient will feel a comfortable tingling sensation, which may also be called paresthesia, in the same region as their pain, which overlaps with the region of pain and/or otherwise reduces pain. Further, if the big toe is the location of an amputation surgery, the system, device, and method may identify and stimulate a target peripheral nerve such as the deep branch of the peroneal nerve sufficiently so as to generate a comfortable tingling sensation or paresthesia before the amputation surgery, which overlaps with the area of potential development of chronic pain and prevents the subsequent development of chronic pain.

The system, device, method, or instructions for use of the system, device, or method may also be used to prevent and/or relieve other effects that patients may experience before, during, or after an amputation surgery. Non-limiting examples of such effects include depression, disability due to pain, pain interference with function or daily activities of living. The present system may therefore improve function and other qualities of life.

It is to be appreciated that the sensation could be described with other words such as buzzing, thumping, etc. Evoking comfortable sensations or paresthesia in the region of pain confirms correct placement of the lead(s) 112 and indicates stimulus intensity is sufficient to reduce pain. Inserting the lead(s) 112 percutaneously may allow the lead(s) 112 to be placed quickly and easily. Placing the lead(s) 112 in a peripheral location, i.e., tissue, where it is less likely to be dislodged, may address lead migration problems of spinal cord stimulation that may otherwise cause decreased coverage of comfortable sensations or paresthesia, decreased pain relief, and the need for frequent patient visits for reprogramming.

Placing the lead(s) 112 percutaneously in tissue with the electrode(s) in proximity to but spaced away from the targeted peripheral nerve may also minimize complications related to lead placement and movement. In a percutaneous system, the lead(s) 112 may be configured as a coiled fine wire electrode lead. This configuration may be used because it is minimally-invasive and well suited for placement in proximity to a peripheral nerve. The lead(s) 112 may be sized and configured to withstand mechanical forces and resist migration during long-term use, particularly in flexible regions of the body, such as the shoulder, elbow, and knee.

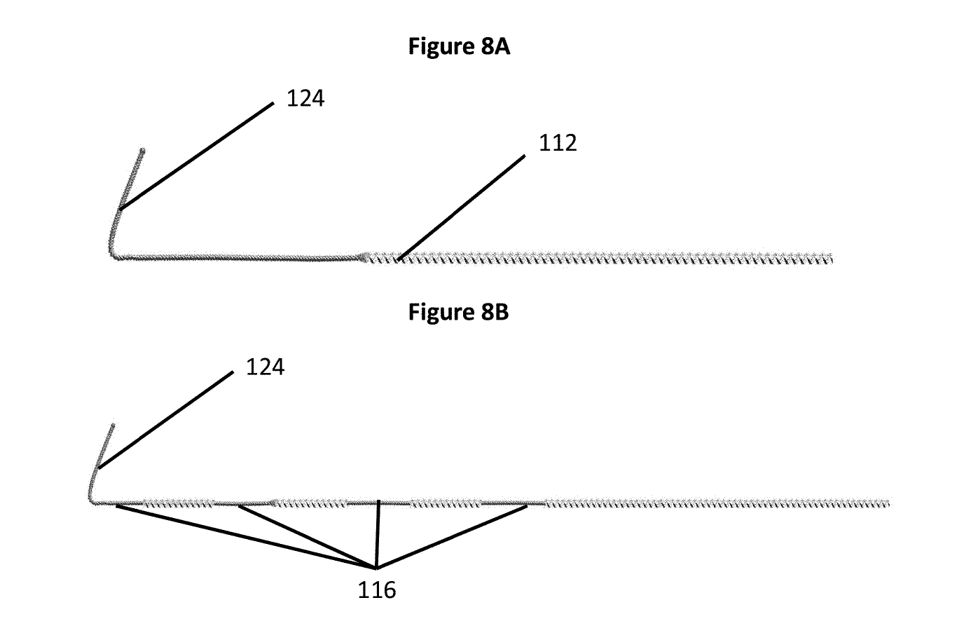

The lead(s) 112 may include a fine wire electrode 116 (as FIGS. 6A and 6B show), paddle electrode, intramuscular electrode, or general-purpose electrode, inserted via a needle introducer or surgically implanted in proximity of a targeted peripheral nerve. Once proper placement of the electrode 116 is confirmed, the needle introducer may be withdrawn (as FIG. 6C shows), leaving the electrode 116 in place. Stimulation may also be applied through a penetrating electrode, such as an electrode array comprised of any number (i.e., one or more) of needle-like electrodes that may be inserted into the target site. In both cases, the lead may be placed using a needle-like introducer 120, allowing the lead(s) 112 and electrode 112 placement to be minimally invasive. In a representative embodiment, the lead(s) 112 may include a thin, flexible component made of a metal and/or polymer material. By “thin,” it is contemplated that the lead(s) 112 may not be greater than about 0.75 mm (0.030 inch) in diameter. However, the present teachings are not limited to such dimensions. Any appropriate lead may be utilized without departing from the present teachings. The lead(s) 112 may also include one or more coiled metal wires with an open or flexible elastomer core. The wire may be insulated, e.g., with a biocompatible polymer film, such as polyfluorocarbon, polyimide, or parylene. The lead may be electrically insulated everywhere except at, for example, one (monopolar), or two (bipolar), or three (tripolar) conduction locations near its distal tip (FIG. 8). A non-limiting example of such is disclosed in U.S. Pat. No. 5,366,493, which is hereby incorporated by reference. Each of the conduction locations may be connected to one or more conductors that may run the length of the lead(s) 112 and extension cable or cables 113 (with lead 112 exiting the skin at point 112S) used to connect the lead to an external pulse generator/stimulator (see FIG. 6C) or a portion thereof. The conductor may provide electrical continuity from the conduction location through the lead to an external pulse generator or stimulator (see FIG. 6C).

The conduction location or electrode 116 may include a de-insulated area of an otherwise insulated conductor that may run the length of an entirely insulated electrode or a portion thereof. The de-insulated conduction region of the conductor may be formed differently, e.g., it may be wound with a different pitch, or wound with a larger or smaller diameter, or molded to a different dimension. The conduction location or the electrode may include a separate material (e.g., metal or a conductive polymer) exposed to the body tissue to which the conductor of the wire is bonded.

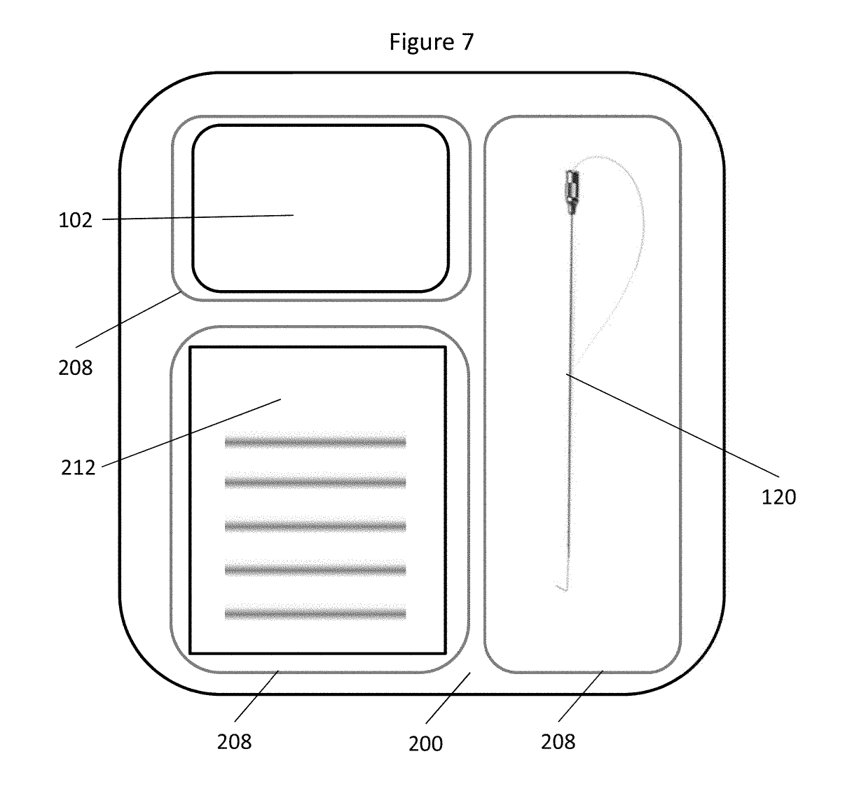

The lead(s) may be provided in a sterile package 200 (see FIG. 7), and may be pre-loaded in an introducer needle 120. Alternatively, the lead may be introduced via the same needle that is used to inject anesthetic or analgesics during peripheral nerve blocks, which are often used in post-amputation surgery. The sterile package 200 may take various forms and the arrangement and contents of the sterile package 200 may be as appropriate related to the use thereof. The sterile package 200 may include a sterile, wrapped assembly. The sterile package 200 may include one or more interior trays 208 made from any appropriate material, e.g., from die cut cardboard, plastic sheet, or thermo-formed plastic material, which may hold the contents. The sterile package 200 may include any appropriate number of interior trays 208, including, without limitation, one, two, three, four, etc., including, without limitation the three such interior trays 208 shown. The sterile package 208 may also desirably include instructions 212 for use regarding using the contents of the sterile package 200 to carry out the lead(s) location and placement procedures, as will be described in greater detail below.

Embodiments of the lead 112 shown in FIG. 6A may include a minimally invasive coiled fine wire lead 112 and electrode 116. The lead(s) 112 may possess mechanical properties in terms of flexibility and fatigue life that provide an operating life free of mechanical and/or electrical failure, taking into account the dynamics of the surrounding tissue (i.e., stretching, bending, pushing, pulling, crushing, etc.)—examples of which are described above. The material of the electrode(s) 116 may discourage the in-growth of connective tissue along its length or an applicable portion thereof, so as not to inhibit its withdrawal at the end of its use. However, it may be desirable to encourage the in-growth of connective tissue at the distal tip of the electrode 116, to enhance its anchoring in tissue.

The electrode 116 may also include, at its distal tip, an anchoring element 124. In the illustrated embodiments, the anchoring element 124 may take the form of a simple barb or bend (see also FIG. 8).

The anchoring element 124 may be sized and configured so that, when in contact with tissue, it takes purchase in tissue, to resist dislodgement or migration of the electrode 116 out of the correct location in the surrounding tissue. Desirably, the anchoring element 124 may be prevented from fully engaging body tissue until after the electrode 116 has been correctly located and deployed.

Alternatively, or in combination, stimulation may be applied through any type of nerve cuff (spiral, helical, cylindrical, book, flat interface nerve electrode (FINE), slowly closing FINE, etc.), paddle (or paddle-style) electrode lead, cylindrical electrode lead, echogenic needle (i.e., visible under ultrasound) and/or other lead that is surgically or percutaneously placed within tissue at the target site. The lead 112 may include an anchoring element 124 in the form of a simple barb or bend to maintain the lead 112 in the tissue at the target site at an appropriate therapeutic distance from the targeted peripheral nerve.

In a preferred embodiment, the lead(s) 112 may exit through the skin and connect with one or more external electrical stimulation devices 102 (this approach is shown in FIG. 6C). Further, the lead(s) 112 may be connected as needed to internal and external coils for RF (Radio Frequency) wireless telemetry communications or an inductively coupled telemetry to control the stimulation device(s). In another embodiment, the lead(s) 112 may connect with one or more implanted pulse generators located some distance (remote) from the electrode 116, or an implanted pulse generator may be integrated with an electrode(s) (not shown), eliminating the need to route the lead 112 subcutaneously to the implanted pulse generator.

The introducer needle 120 may be made from conductive and/or non-conductive materials, with its stimulating portion of the lead 112 (e.g., the electrode 116) housed inside. This stimulating portion may protrude from the end of the introducer needle 120 itself so as to come into contact with the body tissue in which the lead 112 is inserted. The distal end of the electrode 116 may also protrude from the end of the introducer needle 120 in the same manner. The introducer needle 120, lead 112, and/or electrode 116 may then be connected to an electrical stimulation device, such as an external pulse generator (e.g., see FIG. 6A) during/as a part of the introducer/implantation process. Applying stimulating current through the electrode 116 while it is housed within the introducer needle 120 may provide a close approximation to the response that the electrode 116 will provide when it is deployed at the location of the introducer needle 120 because stimulation will be delivered through the same conductive portion of the electrode 116.

The introducer needle 120 may be sized and configured to be bent by hand prior to its insertion through the skin. This may allow the physician to place the lead(s) 112 in a location that is not in an unobstructed straight line with the insertion site. The construction and materials of the introducer needle 120 may allow bending without interfering with the deployment of the lead(s) 112 and withdrawal of the introducer needle 120, leaving the lead(s) 112 in the tissue.

Representative lead insertion techniques will now be described to place an electrode 116 and lead 112 in a desired location in tissue in electrical proximity to but spaced away from a peripheral nerve. In the preferred embodiment, a single lead 112 may be placed to provide pain relief by targeting the peripheral nerve that innervates the region of pain. It is this placement that may make possible the stimulation of the targeted nerve or peripheral nerves with a single lead 112 to provide pain relief. It may also be desirable to place multiple leads to target a single peripheral nerve, for example by placing one lead medial to and one lead lateral to (or one lead superficial to and one lead deep to) a targeted peripheral nerve to activate target nerve fibers distributed throughout the nerve rather than preferentially activating nerve fibers on one side of the nerve. It may also be desirable to place multiple leads to target multiple peripheral nerves (using one or multiple leads for each targeted nerve) to provide pain relief in multiple regions of pain or in a region of pain that spans the regions of innervation of multiple peripheral nerves or branches of peripheral nerves.

To determine the optimal placement for the lead 112, test stimulation may be delivered through a test needle, including but not limited to those described in U.S. patent application Ser. No. 15/388,128, filed on Dec. 22, 2016 and incorporated by reference herein (along with any other applications or documents identified and incorporated by reference in that original document). These and other test needles may include one or more electrodes and/or be made from conductive material(s). The test needle may be selectively, electrically insulated to create a stimulating surface or surfaces on or near its distal tip, whereas the proximal tip has a hub, plug, and/or other connection mechanisms to allow the test needle to operate in concert with an external stimulation device, such as a pulse generator. These types of test needles may be used because they may be easily repositioned until the optimal location to deliver stimulation and generate paresthesia is determined. Alternatively, in some embodiments, the introducer or test needle may carry with the stimulation lead/electrode.

At least one lead(s) 112 may be placed in tissue near a targeted peripheral nerve (e.g., 0.1 cm to 10 cm with a preferred range between 0.5 cm and 3.0 cm remote from the targeted peripheral nerve). The lead(s) 112 may be inserted via the introducer needle 120 in any appropriate manner, which may be in some exemplary embodiments similar in size and shape to a hypodermic needle. The introducer needle 120, however, may be any size. By way of a non-limiting example, the introducer needle 120 may range in size from 17 gauge to 26 gauge. Before inserting the introducer needle 120, the insertion site may be cleaned with a disinfectant (e.g., Betadine, 2% Chlorhexidine/80% alcohol, 10% povidone-iodine, or similar agent). A local anesthetic(s) may be administered topically and/or subcutaneously to the area in which the electrode 116 and/or introducer needle 120 will be inserted.

The position of the electrode(s) 116 on the test needle and/or on the lead 112 itself may be checked by visualizing the test needle or introducer needle 120 using imaging techniques, such as ultrasound, fluoroscopy, or X-rays. Following placement of the lead(s) 112, the portion of the lead(s) 112 which exit the skin may be secured to the skin using covering bandages and/or adhesives or any other appropriate method and mechanism, as shown in FIG. 6D.

Electrical stimulation may be applied to the targeted peripheral nerve during and after placement of the electrode 116. Electrical stimulation may be applied to the targeted peripheral nerve through the electrode 116 while the patient describes the sensations and location of the sensations that are generated by the stimulation. The intensity of the stimulation may be increased or decreased to change the sensations and the location of sensations reported by the patient. When this method is used during the placement of the electrode 116, the sensations and location of sensations reported by the patient may also be modified by changing the location of the electrode 116 placement near the targeted peripheral nerve (e.g., 0.1 cm to 10 cm with a preferred range of 0.5 cm to 3.0 cm remote from the targeted nerve). This method may be used to determine whether stimulation of the targeted peripheral nerve can generate comfortable sensations or paresthesia that overlap with the region of pain and/or reduce pain.

In a percutaneous system (as FIGS. 6A to 6C) shown, the lead 112 may be percutaneously placed near the targeted peripheral nerve and exit at a skin puncture site. A trial or screening test may be conducted in any appropriate clinical setting (e.g., an office of a clinician, a laboratory, a procedure room, an operating room, an intensive care unit, an acute rehabilitation facility, a subacute rehabilitation facility, etc.). During the trial, the lead 112 may be coupled to an external pulse generator and temporary percutaneous and/or surface return electrodes, to confirm paresthesia coverage and/or pain relief of the painful areas.

If the clinical screening test is successful, the patient may proceed to treatment with an external pulse generator or external electrical stimulation device 102 (as shown in FIG. 6C) and temporary percutaneous and/or surface return electrodes 114. The treatment period may range from minutes to hours to days to weeks to months. By way of a non-limiting example, the treatment period may be between approximately 21 and 30 days, or between approximately 56 and 60 days.

Electrical stimulation may be applied between the lead 112 and return electrode(s) 116 (monopolar mode). Regulated current may be used as a type of stimulation, but other type(s) of stimulation (e.g., non-regulated current such as voltage-regulated) may also be used. Multiple types of electrodes may be used, such as surface, percutaneous, and/or implantable electrodes.

In embodiments of a percutaneous system, the surface electrode(s) 114 may serve as the anode(s) (or return electrode(s)). The surface electrodes 114 may be a standard shape or they may be modified as appropriate to fit the contour of the skin. When serving as a return electrode(s) 114, the location of the electrode(s) 116 may not be critical and may be positioned anywhere in the general vicinity.

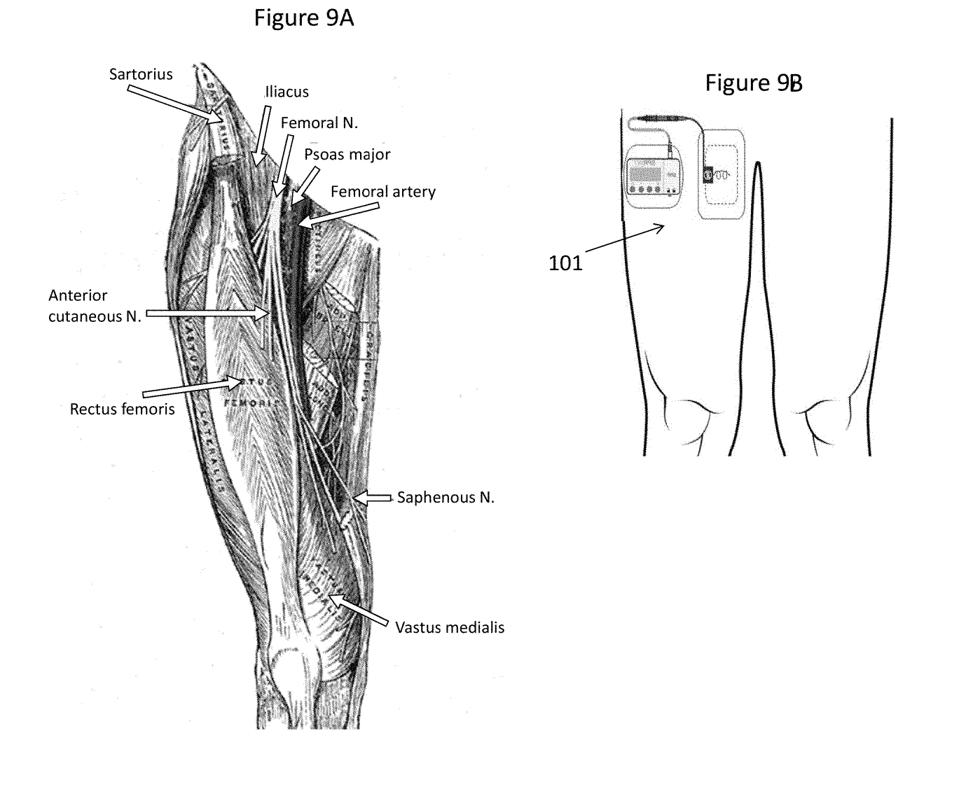

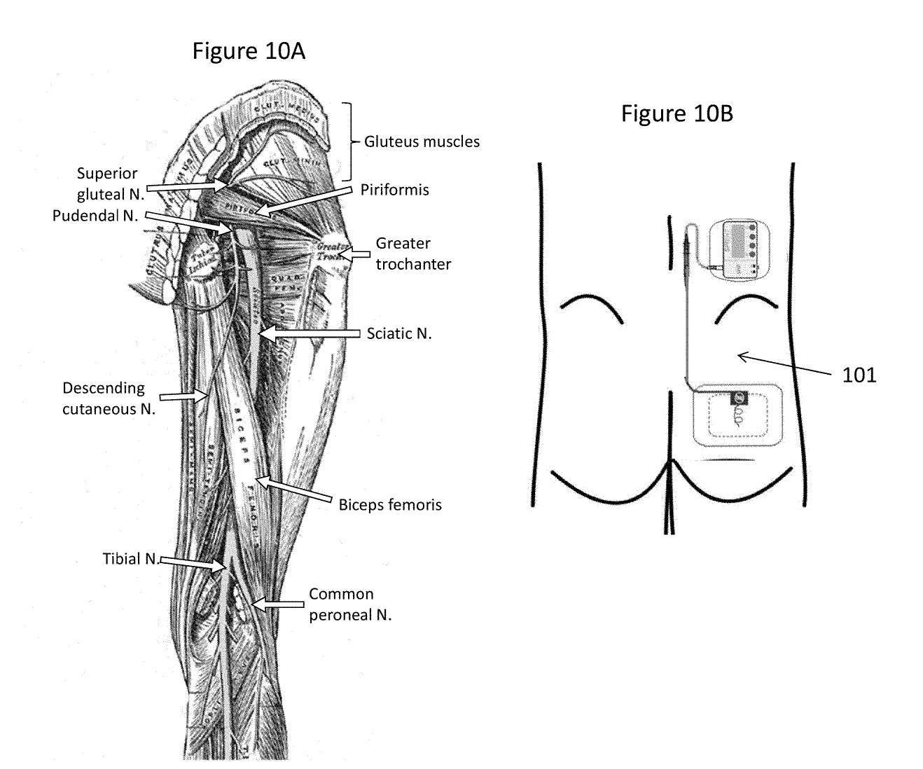

The electrode 116 and lead 112 may be placed via multiple types of approaches. By way of a non-limiting example, when the targeted peripheral nerve includes one or more nerves of the lumbar plexus or sacral plexus, the approach may be either an anterior (shown in FIG. 9) or a posterior approach (shown in FIG. 10). This may be similar to those used for regional anesthesia of the same-targeted peripheral nerve, except that the approach may be used for placement through an introducer of stimulation lead(s) in electrical proximity to but spaced away from a peripheral nerve (e.g., any combination of whole integers to form lower and upper ranges between 1 millimeter and 100 millimeters, with a preferred range between 5 millimeters and 30 millimeters), and not for regional anesthesia. Unlike regional anesthesia, the approach to nerves of the lumbar plexus or sacral plexus may not involve the application of anesthesia to the nerve, and, when the introducer needle 120 is withdrawn, the lead(s) 112 may be left behind to desired stimulation of the target peripheral nerve.

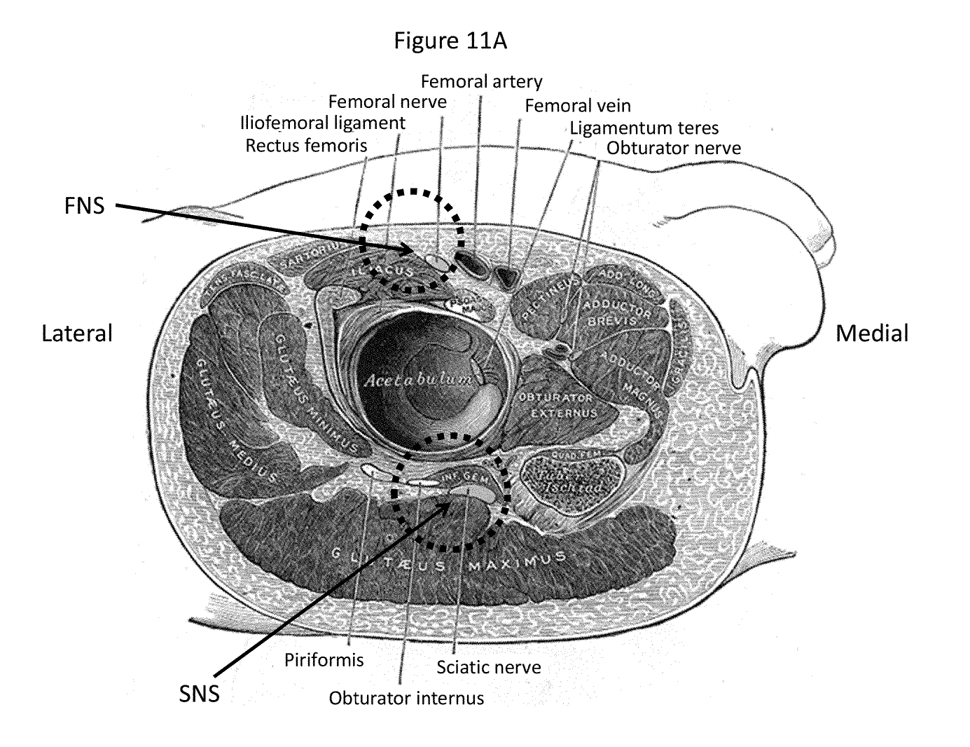

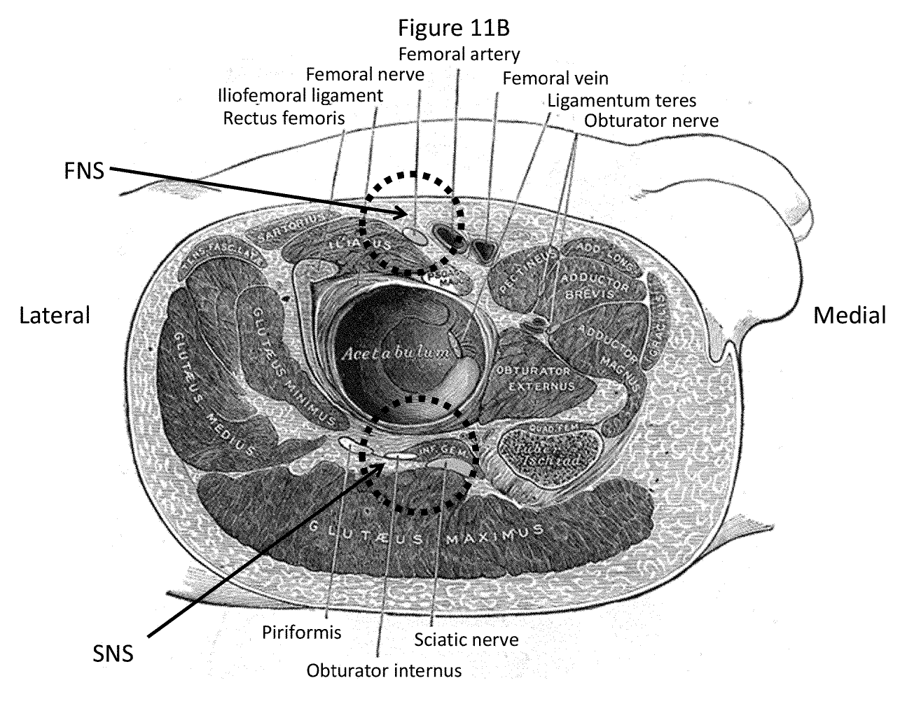

In other embodiments, when the targeted peripheral nerve includes the sciatic nerve (see FIGS. 10 and 11), the introducer needle(s) 120 and/or lead(s) 112 may be directed towards the sciatic nerve using a posterior approach, such as the transgluteal approach or subgluteal approach, which are both used in regional anesthesiology. This approach may allow placement of a lead near a targeted peripheral nerve with a simple, quick (e.g., less than 10 minutes) procedure. It may also be desirable to place multiple leads to target a single peripheral nerve to provide pain relief in the entire region of pain, or to place multiple leads to target multiple peripheral nerves (using one or multiple leads for each targeted nerve) to provide pain relief in multiple regions of pain or in a region of pain that spans the regions of innervation of multiple peripheral nerves or branches of peripheral nerves.

The landmarks for the transgluteal approach may include the greater trochanter and the posterior superior iliac spine. The introducer needle 120 may be inserted distal to the midpoint between the greater trochanter and the posterior iliac spine (e.g., approximately 2 cm to 6 cm distal, preferably 4 cm, in a preferred embodiment). As a non-limiting example of patient positioning, the patient may be in a lateral decubitus position and tilted slightly forward. The landmarks for the subgluteal approach may include the greater trochanter and the ischial tuberosity. The introducer needle 120 and, by extension, either or both of the test electrodes and the lead 112 and its associated electrode(s) 116, are inserted distal (e.g., approximately 2 cm to 6 cm, preferably 4 cm, in the preferred embodiment) to the midpoint between the greater trochanter and the ischial tuberosity.

By way of a non-limiting example, when the targeted peripheral nerve includes the femoral nerve (see FIG. 11), percutaneous lead(s) 112 may be directed towards the femoral nerve using an anterior approach. The landmarks may include the inguinal ligament, inguinal crease, and femoral artery. The subject may be in the supine position with the ipsilateral extremity slightly (approximately 10 to 20 degrees) abducted. The introducer needle 120 may be inserted near the femoral crease but below the inguinal crease and approximately 1 cm lateral to the pulse of the femoral artery.

The size and shape of tissues, such as the buttocks, surrounding the target nerves may vary across subjects, and the approach may be modified as appropriate to accommodate various body sizes and shapes to access the target nerve. As non-limiting examples, the angle of needle insertion, depth of needle insertion, proximal or distal location of insertion on an extremity, or location of lead or electrode placement relative to certain fascial planes, muscular or bony landmarks, or the targeted peripheral nerve may be modified as appropriate.

Introducer needle 120 placement may be guided by the individual's report of stimulus-evoked sensations (paresthesia) as the introducer needle 120 is placed during test stimulation.

More than a single lead 112 may be placed around a given peripheral nerve, using either an anterior approach (e.g., femoral nerve) or a posterior approach (e.g., sciatic nerve). For example, multiple leads may be used to target a single peripheral nerve if one lead produces only partial coverage of the region of pain and the remainder of the region of pain is in the innervation area of the same targeted nerve. Multiple leads may be placed around the same nerve, for example by placing one lead medial to and one lead lateral to, or one lead superficial to and one lead deep to, or in some other pattern or arrangement around the targeted nerve. It may also be desirable to place multiple leads to target multiple peripheral nerves or branches of peripheral nerves, for example to provide pain relief in one or more multiple regions of pain that exist in or span the regions of innervation of multiple peripheral nerves or branches of peripheral nerves.

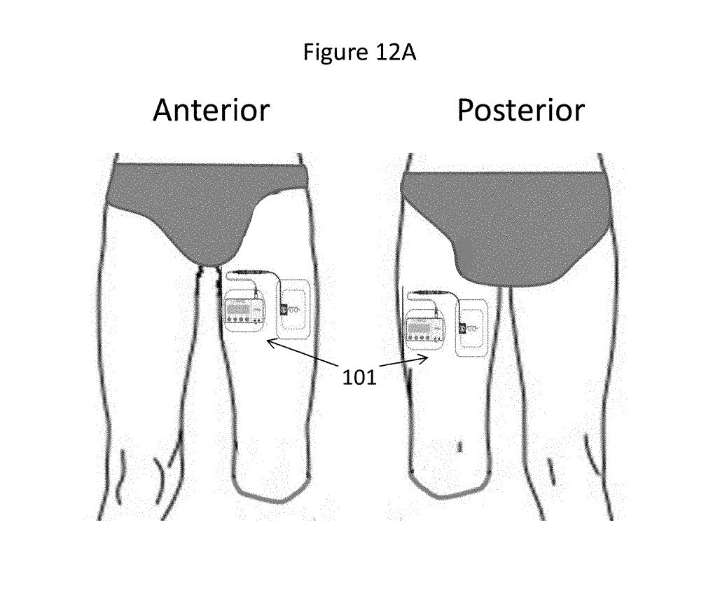

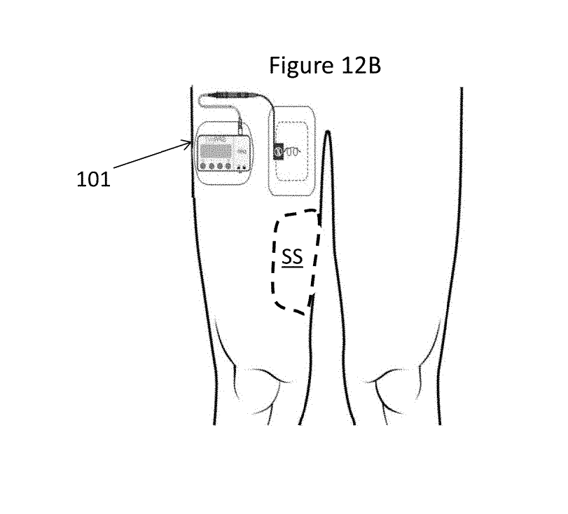

As FIGS. 9A, 9B (anterior approach, e.g., femoral nerve), 10A, 10B (posterior approach, e.g., sciatic nerve), and FIG. 12A, 12B show, the lead may be coupled to the external pulse generator or external electrical stimulation system 101 worn, e.g., on a belt, for a temporary stimulation regime in the preferred embodiment. In this arrangement, the lead may be covered with a bandage, and a surface electrode 114 may serve as a return electrode, as illustrated in FIG. 6D. It may also be desirable to replace the external/percutaneous system shown in FIGS. 9A, 9B and 10A, 10B with an implanted system using an implanted pulse generator and tunneled leads. In this arrangement, the case of the implanted pulse generator may be formed from a conductive material connected to the stimulation hardware housed within so as to serve as the return electrode.

Control of the electrical stimulation device (e.g., the pulse generator) and its stimulation parameters may be provided by one or more external controllers. Alternatively, a controller may be integrated with the external electrical stimulation device 102. The implanted pulse generator external controller (i.e., clinical programmer) may be a remote unit that uses RF (Radio Frequency) wireless telemetry communications (rather than an inductively coupled telemetry) to control the implanted pulse generator. The electrical stimulation device 102 may use passive charge recovery to generate the stimulation waveform, regulated voltage (e.g., 10 mV to 20 V), and/or regulated current (e.g., about 10 mA to about 50 mA). Passive charge recovery may be one method of generating a biphasic, charge-balanced pulse as desired for tissue stimulation without severe side effects due to a DC component of the current.

The neurostimulation pulse may be monophasic (anodic or cathodic), biphasic, and/or multi-phasic. In the case of the biphasic or multi-phasic pulse, the pulse may be symmetrical or asymmetrical. Its shape may be rectangular or exponential or a combination of rectangular and exponential waveforms. The pulse width of each phase may range between e.g., about 0.1 μsec. to about 1.0 sec., as non-limiting examples. Changes in the pulse width of each phase may change the perception of the stimulation, comfort of the sensations from stimulation, or location of sensations from the stimulation that are sensed, described, or reported by the patient.

Pulses may be applied in continuous or intermittent trains (i.e., the stimulus frequency changes as a function of time). In the case of intermittent pulses, the on/off duty cycle of pulses may be symmetrical or asymmetrical, and the duty cycle may be regular and repeatable from one intermittent burst to the next or the duty cycle of each set of bursts may vary in a random (or pseudo random) fashion. Varying the stimulus frequency and/or duty cycle may assist in warding off habituation because of the stimulus modulation. Habituation of a patient to the sensations from stimulation can result in a decrease in the perceived intensity of stimulation. Avoiding habituation may be desirable to ensure that patient sensations are maintained at a perceived intensity that has a therapeutic effect.

The stimulating frequency may range from e.g., about 1 Hz to about 300 Hz. The frequency of stimulation may be constant or varying. In the case of applying stimulation with varying frequencies, the frequencies may vary in a consistent and repeatable pattern or in a random (or pseudo random) fashion or a combination of repeatable and random patterns. In the case of applying stimulation with constant frequency, the pattern of pulses may be a repeated train of pulses with regular intervals selected from a range (e.g., 1 pulse per second up to about 300 pulses per second).

In an embodiment, the stimulator may be set within a range of intensities (e.g., any combination of two whole integers between 1.0 and 30.0 mA and, separately, between 10 and 200 microseconds, so as to form lower and upper limits for each) and frequencies (e.g., any combination of two whole integers between 1 and 100 Hertz, again forming lower and upper limits to the ranges contemplated and expressly disclosed herein).

The distance between the targeted nerve and the lead/electrode must be sufficient to activate the targeted nerve (as noted above, preferably falling between 5.0 to 30 mm but also including any combination of numbers taken to the hundredths decimal place between 0.001 and 100.00 millimeters) away from the targeted peripheral nerve. As non-limiting examples, the lead could be placed at 0.03 mm, 6.23 mm, or 99.99 mm away from the targeted nerve, so long as the nerve is activated and the lead avoids physical contact of any portion of the electrode (i.e., the exposed, stimulating surface) with the targeted nerve. As noted throughout, and particularly in this paragraph, any disclosed range or combination of ranges may display inherent advantages in comparison to the previous teachings in this field.

If the stimulus intensity is too great, it may generate muscle twitch(es) or contraction(s) sufficient to disrupt correct placement of the lead 112. If stimulus intensity is too low, the lead 112 may be advanced too close to the targeted peripheral nerve (beyond the optimal position), possibly leading to incorrect guidance, mechanically evoked sensation (e.g., pain and/or paresthesia) and/or muscle contraction (i.e. when the lead touches the peripheral nerve), inability to activate the target nerve fiber(s) without activating non-target nerve fiber(s), improper placement, and/or improper anchoring of the lead 112 (e.g., the lead 112 may be too close to the nerve and no longer able to anchor appropriately in the muscle tissue).

Patient sensation may instead be used to indicate electrode 112 location relative to the targeted peripheral nerve as indicator(s) of lead placement (distance from the peripheral nerve to electrode contact). Any combination of stimulus parameters that evoke sensation(s) may be used. The stimulation parameters may include, but are not limited to frequency, pulse duration, amplitude, duty cycle, patterns of stimulus pulses, and waveform shapes. Some stimulus parameters may evoke a more desirable response (e.g., more comfortable sensation, or a sensation that may be correlated with or specific to the specific target nerve fiber(s) within the targeted peripheral nerve. As a non-limiting example, higher frequencies (e.g., 100 Hz or 12 Hz) may evoke sensation(s) or comfortable paresthesia(s) in the region(s) of pain or in alternate target region(s).

While stimulation is being applied, the lead 112 (non-limiting examples of the lead could include a single or multi-contact electrode that is designed for temporary (percutaneous) or long-term (implant) use or a needle electrode (used for in-office testing only), e.g., see FIG. 8) may be advanced (e.g., slowly advanced) towards the targeted peripheral nerve until the desired indicator response (e.g., patient paresthesia sensation, pain relief, and/or distance from the nerve) is obtained. The intensity may then be decreased (e.g., gradually decreased) as the lead 112 is advanced (e.g., advanced slowly) closer to the targeted nerve until the desired indicator response(s) may be obtained at smaller intensity(ies) within a target range (e.g., 0.1-1.0 mA (or 0.09-39 mA, or 0.009-199 mA), 100-300 μs (or 40-1000 μs, or 1-10,000 μs)).

The appropriate electrode spacing from a targeted nerve may depend on various factors, and similar stimulation settings may evoke different responses from different peripheral nerves or from the same nerve but at different locations along the nerve. Differences between patients also evoke different responses, even if the electrode 116 is spaced at similar distances. Thus, electrode spacing from the nerve may be about 10 to about 20 millimeters for one target nerve at a given stimulation intensity while the spacing may be about 20 to about 40 millimeters for a second target nerve at the same stimulation intensity. As the distance between the electrode and the target nerve increases the effects of the stimulation at the target nerve may decrease.

If specific response(s) (e.g., desired response(s) and/or undesired response(s)) may be obtained at a range of intensities that are too low, then the electrode 112 may be located in a non-optimal location (e.g., too close to the target nerve(s)). In such situations, therefore, the clinician may adjust the lead location to change the electrode(s) location(s) until the appropriate responses are achieved from the patient.

The stimulus intensities may be a function of many variables. The stimulus intensities set forth herein are meant to serve as non-limiting examples only, and may need to be scaled accordingly. As a non-limiting example, if electrode shape, geometry, or surface area were to change, then the stimulus intensities may need to change appropriately. For example, if the intensities were calculated for a lead with an electrode surface area of approximately 20 mm², then the intensities may need to be scaled down accordingly to be used with a lead having an electrode surface area of 0.2 mm² because a decrease in stimulating surface area may increase the current density, increasing the potential to activate excitable tissue (e.g., target and non-target nerve(s) and/or fiber(s)). Alternatively, if the intensities were calculated for a lead with an electrode surface area of approximately 0.2 mm², then the intensities may need to be scaled up to be used with a lead with an electrode surface area of 20 mm². Alternatively, stimulus intensities may need to be scaled to account for variations in electrode shape or geometry (between or among electrodes) to compensate for any resulting variations in current density. In a non-limiting example, the electrode contact surface area may be between approximately 0.1-20 mm², 0.01-40 mm², or 0.001-200 mm². In a further non-limiting example, the electrode contact configuration may include one or more of the following characteristics: cylindrical, conical, spherical, hemispherical, circular, triangular, trapezoidal, raised (or elevated), depressed (or recessed), flat, and/or borders and/or contours that are continuous, intermittent (or interrupted), and/or undulating.

Stimulus intensities may need to be scaled to account for biological factors, including but not limited to patient body size, weight, mass, habitus, age, and/or neurological condition(s). As a non-limiting example, patients that are older, have a higher body-mass index (BMI), and/or neuropathy (e.g., due to diabetes) may need to have stimulus intensities scaled higher (or lower) accordingly.

As mentioned above, if the lead 112 is too far away from the targeted peripheral nerve, then stimulation may be unable to evoke the desired response (e.g., comfortable sensation(s) (or paresthesia(s)), and/or pain relief) in the desired region(s) at the desired stimulus intensity(ies). If the lead 112 is too close to the targeted peripheral nerve, then stimulation may be unable to evoke the desired response(s) (e.g., comfortable sensation(s) (or paresthesia(s)), and/or pain relief) in the desired region(s) at the desired stimulus intensity(ies) without evoking undesirable response(s) (e.g., unwanted and/or painful sensation(s) (or paresthesia(s)), increase in pain, and/or generation of additional pain in related or unrelated area(s)). In some cases, it may be difficult to locate the optimal lead placement (or distance from the targeted peripheral nerve) and/or it may be desirable to increase the range of stimulus intensities that evoke the desired response(s) without evoking the undesired response(s) so alternative stimulus waveforms and/or combinations of leads and/or electrode contacts may be used. In the preferred embodiment, a biphasic, charge-balanced pulse may be generated for tissue stimulation, however, non-limiting examples of alternative stimulus waveforms may include the use of a pre-pulse to increase the excitability of the target fiber(s) and/or decrease the excitability of the non-target fiber(s).

This stimulation may be used pre-operatively or intra-operatively to limit or prevent post-operative pain. Those skilled in the art will recognize that, for simplicity and clarity, the full structure and operation of all devices and processes suitable for use with the present teachings are not being depicted or described herein. The present disclosure contemplates combining the various features described above in any manner and is not limited solely to the combinations described above.

III. Example of a Method of Use

Following a transfemoral (above-knee) lower limb amputation (TFA), the majority of patients experience moderate to severe acute pain, and a lesser number continue to experience moderate to severe subacute pain and chronic pain. Acute and subacute postoperative pain may limit early recovery and rehabilitation. The patients experience different types of pain, including nociceptive, inflammatory, and neuropathic pain. The mid-thigh is innervated by the femoral, lateral femoral cutaneous, obturator, and the sciatic nerves. Anesthetic block of these nerves individually or as a group may reduce acute pain following a TFA. Adequate treatment of acute and subacute pain may also prevent the development of debilitating chronic pain resulting from TFA. Accordingly, electrical stimulation of nerves that innervate, or portions of which innervate, a portion of the body (specifically a limb or joint) to undergo amputation surgery, where such stimulation occurs before, during and/or after amputation surgery, or some combination of those times, may be used to reduce pain and enhance recovery. In this example, if the targeted peripheral nerve includes nerves of the femoral and sciatic nerves and/or their nerve branches, the instruction(s) and method may include:

1) Place the patient in a comfortable and/or appropriate position.

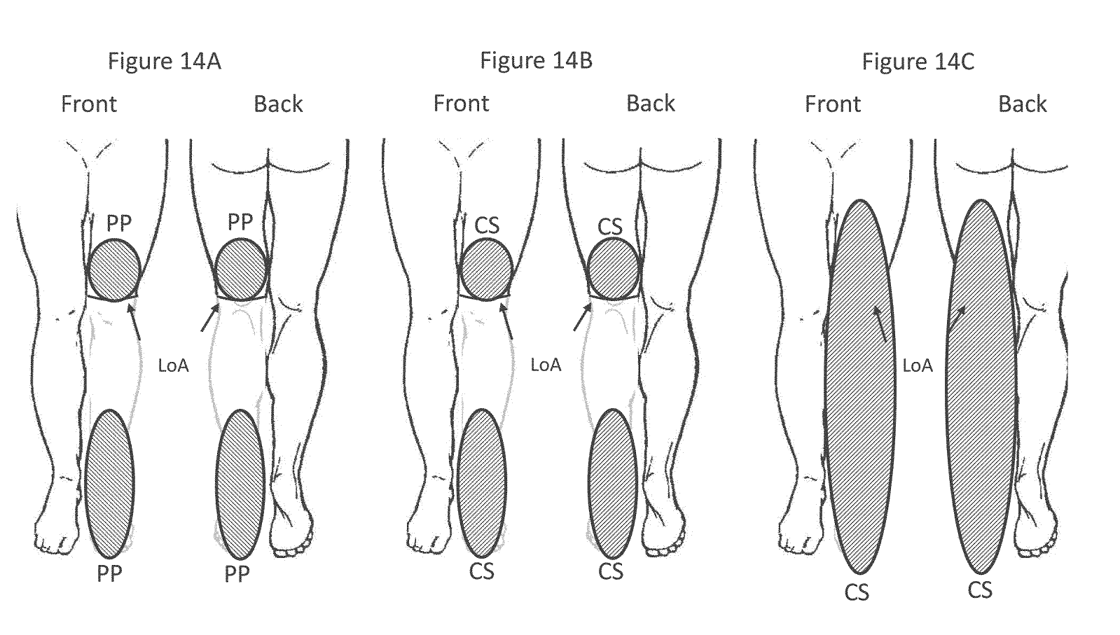

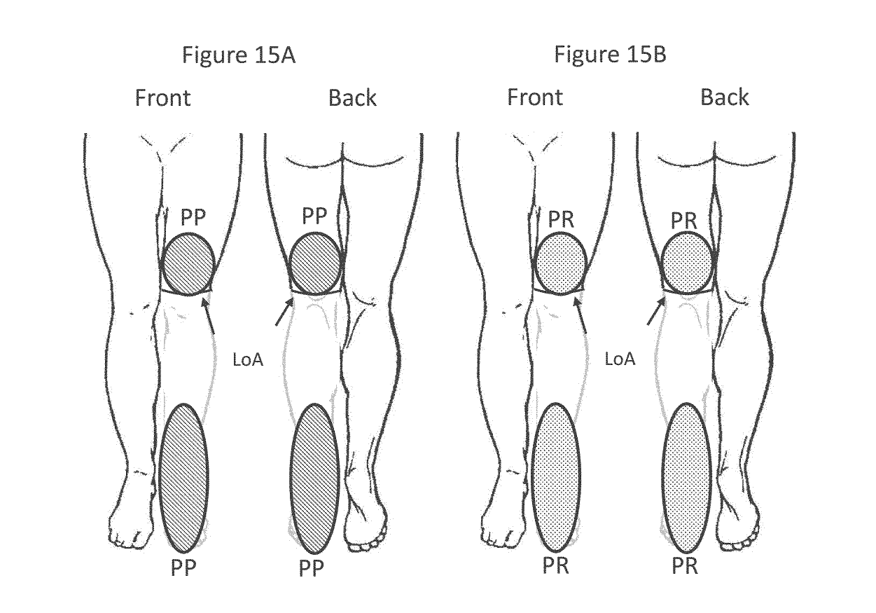

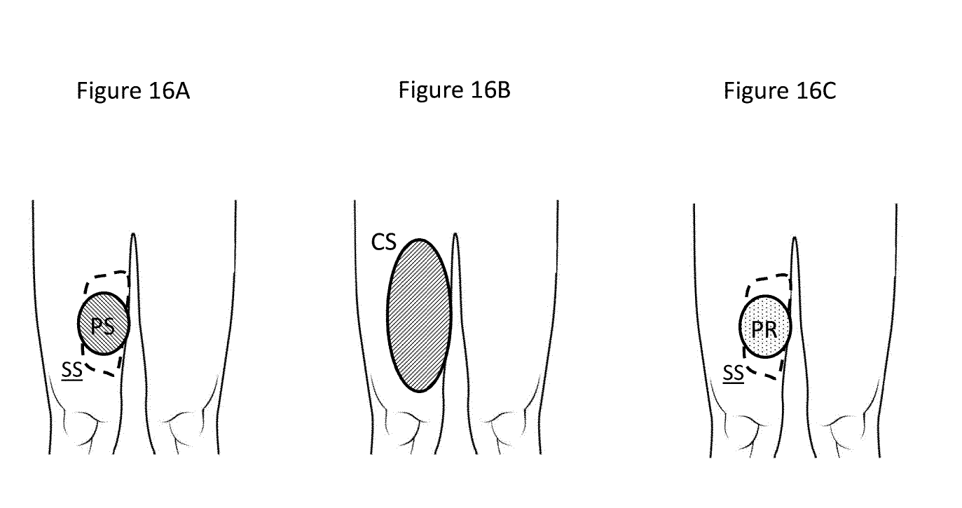

2) Ask the patient to shade their area of pain on a diagram of the body. For example, as shown in FIGS. 14 and 15, the shaded areas indicate where the patient was experiencing pain, where the patient may be experiencing comfortable sensations, and where the patient may be experiencing pain relief (with or without comfortable sensations).

3) Prepare the lead insertion site with antiseptic and local subcutaneous anesthetic (e.g., 2% lidocaine) may be used as well.

4) Locate the site of skin puncture with appropriate landmarks, such as the inguinal crease and femoral artery (for the femoral nerve) and the interior and lateral (ventral) to the midpoint of the line connection greater trochanter and ischial tuberosity (for the sciatic nerve).

5) Insert a sterile percutaneous electrode lead (such as the lead 112 described above) preloaded in the introducer needle (such as that 120 described above) at a predetermined angle based on the landmarks used. The lead 112 may be of any appropriate configuration, such as by way of a non-limiting example, a single fine wire with one lead to target each nerve.

6) Place a surface stimulation return electrode in proximity to the lead insertion site (FIG. 12). The surface electrode may be placed adjacent to the insertion site. Its position is not critical to the therapy and it may be moved throughout the therapy to reduce the risk of skin irritation, but care should be taken to place the electrode distant from the surgical incision to generally avoid infection.

7) Couple the lead 112 to the external pulse generator or external electrical stimulation device 102 and to the return electrode. Set the desired stimulation parameters on the external pulse generator or external electrical stimulation device 102, or through a controller. Test stimulation may be delivered using a current-regulated pulse generator, for example. The external pulse generator may be a battery-powered stimulator, for example.

8) Advance the introducer needle 120 slowly until the subject reports the first evoked sensation in the region experiencing pain. Progressively reduce the stimulus amplitude and advance the introducer needle 120 more slowly until the sensation can be evoked in the painful region at predetermined stimulus amplitude (e.g., 1 mA). Stop the advancement of the introducer needle 120, and increase the stimulus amplitude in small increments (e.g., 0.1 mA) until the stimulation-evoked tingling sensation (paresthesia) expands to overlay the entire region of pain. The electrode 116 may be located at an area to generate maximum paresthesia coverage of the region of pain, as defined by a patient shaded diagram of the body. During stimulation, the patient is asked to estimate how much of the area of pain is covered by paresthesia. For example, as in FIGS. 14 and 15, the shaded regions indicate where the patient experiences paresthesia during stimulation, and where the patient may experience pain relief during stimulation.

9) Withdraw the introducer needle 120, leaving the percutaneous lead 112 in proximity but away from the target nerve. Further, a plurality of leads may be placed percutaneously near or approximately adjacent to the nerves innervating the regions of pain, and stimulation may be applied to determine optimal stimulus parameters and lead locations. Although in some embodiments, only a single lead 112 may be utilized.

10) Cover the percutaneous exit site and lead 112 with a bandage (FIG. 12). A bandage may also be used to secure the external portion of the lead 112 (or an extension cable may be used to couple the lead to the external pulse generator) to the skin. It is expected that the lead 112 will generally take less than 10 minutes to operatively position, although the process may be shorter or longer.

11) The external pulse generator or external electrical stimulation device 102 may be programmed to 100 Hz, 15 μs with amplitude sufficient to generate maximum paresthesia coverage. The parameter may include 100% duty cycle (for both femoral and sciatic) for 24 hours per day. The stimulation may be on for the duration of the acute or subacute pain of the patient. Patients may receive the stimulation therapy for a predetermined time, such as, by way of a non-limiting example, two to four weeks or 28-30 days or 56-60 days.

12) The stimulation intensity may need to be increased slightly during the process due to causes such as habituation or the subject becoming accustomed to sensation. However, the need for increased intensity may be unlikely and usually only occurs after several days to weeks to months as the tissue encapsulates and the subject accommodates to stimulation. It is to be appreciated that the need for increased intensity may happen at any time, which may be due to either lead migration or habituation, but may also be due reasons ranging from nerve damage to plasticity/reorganization in the central nervous system.

13) Prior to insertion of the lead 112 and introducer needle 120, a sterile test needle may be used to deliver stimulation and determine the desired site of insertion.

14) If paresthesia cannot be evoked with the initial lead placement, the introducer needle 120 may be redirected.

15) If stimulation fails to elicit paresthesia in a sufficient region (e.g., >50%) of pain, then a second percutaneous lead may be placed to stimulate the nerves that are not activated by the first lead, i.e., the nerves innervating the region of post-operative pain.

Percutaneous electrical stimulation of nerves innervating the mid-thigh as discussed in the example above may be used to generate paresthesia to provide pain relief for any type of post-op pain following an amputation surgery (e.g., immediate acute phase=0 to 3-5 days; post-acute or subacute phase=3-5 days to 30 days), and/or to prevent the later development of chronic pain. In this approach, one might use the femoral and sciatic nerves, or they may also stimulate the lumbar plexus to target the femoral, obturator, and/or lateral femoral cutaneous nerves. Additionally, there may be an anterior approach as well as a posterior approach to targeting these nerves.

An alternative embodiment may include using a needle electrode/lead and placing it during insertion of needles used during anesthetic peripheral nerve block. Additionally, in a different embodiment the pulse trains may be varied, as varied pulse shapes may improve selectivity of activation of paresthesia fibers versus pain fibers. Percutaneous electrical stimulation of nerves may provide some pain relief as anesthetic block without many of its drawbacks. This therapy may be provided as a temporary therapy or as a permanent implant. Acute pain relief may allow patients to recover sufficiently enabling them to begin rehabilitation. It is generally thought that if 50% paresthesia coverage is achieved, then there is a 70% success rate. Oftentimes after the stimulation therapy, the pain will never return to the patient.

The TFA is discussed herein for the sake of brevity. It, however, is to be understood that the systems and methods may be employed to condition a body before or after any surgical amputation of an extremity, tissue or limb. While stimulation of the femoral and/or sciatic nerves should generally provide relief of pain following an amputation surgery of the leg, more distal peripheral nerves may be targets for amputations related to distal portions of the leg (toe, foot, ankle, e.g.). For arm/hand limb amputation-related pain, nerves near the brachial plexus, near or below the shoulder, elbow, or wrist may be targeted.

In peripheral nerve stimulation, the lead 112 may be placed such that the electrode is in a tissue by which the targeted nerve passes, but stimulation actually relieves pain that is felt distal (downstream) from where the lead 112 is placed. In peripheral nerve stimulation, the lead 112 may be placed in a tissue such that the electrode is conveniently located near a nerve trunk that passes by the lead 112 on the way to or from the painful area. The key is that the lead 112 may be placed in a tissue that is not the target (painful) tissue, but rather a tissue that is located away from the painful region, which is a safer and more convenient location to place the lead 112.

Peripheral nerve stimulation may provide stimulation-generated paresthesia (that ideally overlap with the area of pain) but may not require evoking a muscle contraction to place the lead 112 correctly such that the electrode 116 on the lead 112 is in a preferred location to stimulate the targeted peripheral nerve. The target regions in which pain is felt and which are targeted for generation of paresthesia may not be the same region in which the lead 112 is placed.

Imaging (e.g., ultrasound or an alternate imaging technique, e.g., fluoroscopy) may be used to improve lead 112 placement to ideally locate the electrode near a targeted peripheral nerve. Ultrasound may improve lead 112 placement in the form of increasing the total speed of the procedure. Specifically, ultrasound may shorten the procedure's duration by locating the lead 112 in a more optimal location. Doing so may: improve recruitment of the target fibers in the target nerve and minimize recruitment of non-target fibers in either the target nerve and/or in non-target nerve(s); and minimize risk and/or damage to the patient during placement of the lead by avoiding blood vessels, organs, bones, ligaments, tendons, lymphatic vessels, &/or other structures that may be damaged. One reason that imaging may be useful is that some peripheral nerves are (but do not have to be) located relatively deeply. Alternatively, fluoroscopy may be desirably avoided, thus lessening the cost of the procedure and the risk of radiation exposure.

In the present system and method, the patient may not need to give verbal, written, or other type of feedback or indication of what they feel as the lead is being advanced towards the peripheral nerve if imaging is used to guide lead placement. In addition, any known method for non-verbal communication can be used, including those used by anesthesiologists. This allows the system to be placed in an unconscious patient, e.g., in a sedated patient or intra-operatively. However, patient feedback during lead 112 advancement may improve lead placement in some patients. The patient may indicate sensations during tuning of stimulus intensity. As non-limiting examples, those sensations reported by the patient may include first sensation (minimum stimulus intensity that evokes a sensation), level of comfort, maximum tolerable sensation, pain, and qualities or descriptions of the sensations. Alternatively, if the system is used preoperatively, as there will not be any patient feedback of post-operative pain to guide the paresthesia coverage, the optimal coverage would be a region that is likely to be painful following the amputation surgery (e.g., in the case of a TFA, both the front and back of the mid-thigh).

The region in which the patient perceives stimulation-induced sensations or paresthesia may be an important indicator of the potential success of the therapy. This may help screen potential candidates and may help determine the appropriate stimulation parameters (including but not limited to lead location). Further, such parameters may be adjusted so that the region in which paresthesia is perceived overlaps with the region of pain. For example, the intensity of the electrical stimulation may be increased by increasing the amplitude or pulse duration of the stimulation waveform, thereby activating more targeted fibers in the peripheral nerve and increasing the area or location(s) in which paresthesia is felt until the area includes or overlaps with the region of pain.

As an alternative to using perception of stimulation induced sensations and/or paresthesia, the level of pain or change in the intensity of pain during or due to stimulation may be used to adjust stimulation parameters (including but not limited to lead location). For example, if a patient is experiencing “very high” pain before stimulation, and no sensory or motor responses are evoked during stimulation, if the pain decreases to “low”, the system would be considered satisfactory in the patient.

Stimulation may be delivered so as to provide pain relief without evoking additional sensations or paresthesia. Stimulation may be delivered using sub-threshold (i.e., below the threshold for sensation or perception) or other parameters that provide relief of pain without producing sensations or paresthesia. As a non-limiting example, stimulation may be delivered to evoke a response, such as sensation, paresthesia, motor or muscle response, to guide, confirm, improve, or optimize lead or electrode placement and stimulation parameters, and following placement of the lead(s) and/or electrode(s) stimulation parameters may then be set to provide pain relief without unwanted responses (which may include sensations, paresthesia, motor or muscle activation or contractions). Alternatively, in some scenarios responses such as sensations, paresthesia, and/or muscle activation or contractions may be desirable and/or beneficial and may be produced intentionally as part of, in combination with, and/or simultaneously with the therapy.

Although the embodiments of the present invention have been illustrated in the accompanying drawings and described in the foregoing detailed description, it is to be understood that the present teachings are not to be limited to just the embodiments disclosed, but that the invention described herein is capable of numerous rearrangements, modifications and substitutions without departing from the scope of the claims hereafter. The claims as follows are intended to include all modifications and alterations insofar as they come within the scope of the claims or the equivalent thereof.

The terms “component,” “module,” “system,” “interface,” “platform,” “service,” “framework,” “connector,” “controller,” or the like are generally intended to refer to a computer-related entity. Such terms may refer to at least one of hardware, software, or software in execution. For example, a component may include a computer-process running on a processor, a processor, a device, a process, a computer thread, or the likes. In another aspect, such terms may include both an application running on a processor and a processor. Moreover, such terms may be localized to one computer and/or may be distributed across multiple computers.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the components described above may be combined or added together in any permutation to define the present system and method. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system to alleviate pain following surgical amputation or removal of an extremity, limb, body part, or body tissue, the system comprising:
a coiled wire lead having a thickness and a diameter that are appropriate for percutaneous insertion into a portion of a body that is proximal to or will become part of a residual limb or extremity following surgical amputation;
an electrode formed integrally on the lead, wherein the electrode is positioned: i) inside the body when the system is functioning, and ii) at a therapeutically effective distance from a portion of at least one nerve that will be transected or cut during the surgical amputation; and
an electrical stimulation device operatively coupled to the lead to apply electrical stimulation through the at least one electrode to at least one nerve that will be transected or cut, to create comfortable sensations in a region of residual or phantom pain.

2. The system of claim 1, wherein the electrical stimulation also causes comfortable sensations within the body in an area outside of the region of residual or phantom pain.

3. The system of claim 1, wherein the electrical stimulation occurs proximal to the residual limb or extremity.

4. The system of claim 1, wherein the electrical stimulation includes a first parameter selected from a group consisting of: frequency, pulse duration, amplitude, duty cycle, pattern of stimulus pulses, polarity, a predetermined number of phases, and waveform shape.

5. The system of claim 4, wherein the electrical stimulation includes a second parameter selected from a group consisting of: frequency, pulse duration, amplitude, duty cycle, pattern of stimulus pulses, polarity, a predetermined number of phases, and waveform shape.

6. The system of claim 5, wherein the first and second parameters are identical and each has a defined value and wherein the defined values for the first and second parameters are different.

7. The system of claim 1, wherein the electrical stimulation does not damage the at least one nerve that will be transected or cut.

8. The system of claim 1, wherein the region of residual or phantom pain is at or distal to a location of the surgical amputation, said location selected from a group consisting of: above knee, at knee, below knee, at ankle, foot, toe, above shoulder, at shoulder, above elbow, at elbow, below elbow, at wrist, hand, finger, and breast.

9. The system of claim 1, wherein the region of phantom pain is associated with amputation of appendages or extremities.

10. The system of claim 1, wherein the at least one nerve is a peripheral nerve.

11. The system of claim 1, wherein the at least one nerve that will be transected or cut is selected from a group consisting of a femoral nerve, a sciatic nerve, lateral femoral cutaneous nerve, an obturator nerve, median nerve, radial nerve, ulnar nerve, axillary nerve, musculocutaneous nerve, brachial plexus, lumbar plexus, sacral plexus, intercostal nerve, ilioinguinal nerve, iliohypogastric nerve, and intercostobrachial nerve.

12. A system of claim 11, wherein the at least one nerve selected from the group includes at least one distal branch of the selected nerve or nerves.

13. A system to alleviate pain after an amputation surgery, the system comprising:
at least one electrode placed within a therapeutically effective distance from a peripheral nerve innervating a targeted region of pain resulting from the amputation surgery;
an electrical stimulation device to deliver electrical stimulation through the at least one electrode;
wherein the electrical stimulation activates the peripheral nerve to evoke a comfortable sensation in the expected targeted region of pain but without functional nerve stimulation at a motor point of the peripheral nerve; and
wherein the electrical stimulation device applies electrical stimulation through the at least one electrode after the amputation surgery evoking a second comfortable sensation.

14. The system of claim 13, wherein the electrode is positioned prior to the amputation surgery and wherein electrical stimulation delivered to the peripheral nerve prior to the surgery does not damage the peripheral nerve.

15. The system of claim 14, wherein the electrical stimulation prior to surgery includes a first set of electrical stimulation parameters.

16. The system of claim 15, wherein the electrical stimulation after the amputation surgery includes a set of second electrical stimulation parameters and wherein at least one of the second electrical stimulation parameters is different from at least one of the first electrical stimulation parameters.

17. The system of claim 16, wherein the at least one of the second electrical stimulation parameters includes at least one of frequency, pulse duration, amplitude, duty cycle, pattern of stimulus pulses, polarity, a predetermined number of phases, and waveform shape.

18. The system of claim 17, wherein the at least one electrode is located in tissue that is proximal from the region of pain.

19. The system of claim 13, wherein the amputation surgery includes transtibial or transfemoral lower limb amputation.

20. The system of claim 13, wherein the peripheral nerve is selected from the femoral nerve, the sciatic nerve, and the obturator nerve.

21. The system of claim 13, wherein the electrical stimulation device comprises a pulse generator.

22. The system of claim 21, wherein the pulse generator delivers the electrical stimulation to the peripheral nerve by way of a percutaneous lead attached to the electrode.

23. The system of claim 22, wherein the peripheral nerve, inclusive of any distal branch thereof, is selected from a group consisting of a femoral nerve, a sciatic nerve, lateral femoral cutaneous nerve, an obturator nerve, median nerve, radial nerve, ulnar nerve, axillary nerve, musculocutaneous nerve, brachial plexus, lumbar plexus, sacral plexus, intercostal nerve, ilioinguinal nerve, iliohypogastric nerve, and intercostobrachial nerve.

24. The system of claim 13, wherein the electrical stimulation will not block motor or sensory function of a limb.

25. A kit for treatment of pain from amputation surgery, the kit comprising: a needle insertable into an animal body tissue; a percutaneous lead operatively inserted into the needle, wherein the needle and the percutaneous lead are inserted into an insertion point of the animal body, whereby the needle is removable from the animal body tissue and the percutaneous lead is retained within the animal body; and a system comprising:
at least one electrode placed remote to but within a therapeutically effective distance from a peripheral nerve innervating a targeted region of pain resulting from the amputation surgery;
electrical stimulation delivered through the at least one electrode by an electrical stimulation device;
wherein the electrical stimulation is delivered to the peripheral nerve in the expected targeted region of pain; and
wherein electrical stimulation is then also applied through the at least one electrode by an electrical stimulation device after the amputation surgery.

26. The kit of claim 25, wherein the electrical stimulation causes an area of comfortable sensations and the area of comfortable sensations is compared with the region of expected pain from the amputation surgery.

27. The kit of claim 25, further comprising a test needle.

28. The kit of claim 25, wherein the needle is an introducer needle.

29. The kit of claim 25, further comprising an anchor attached to the percutaneous lead, the anchor configured to operatively retain the percutaneous lead at the insertion point during withdrawal of the needle from the animal body.

30. The kit of claim 25, wherein the amputation surgery is selected from a group consisting of: limb amputation above knee, limb amputation at knee, limb amputation below knee, limb amputation at ankle, full or partial foot amputation, full or partial toe amputation, limb amputation above shoulder, limb amputation at shoulder, limb amputation above elbow, limb amputation at elbow, limb amputation below elbow, limb amputation at wrist, full or partial hand amputation, full or partial finger amputation, and mastectomy or breast amputation.

31. A kit for treatment of pain from removal of an extremity, limb, body part, or body tissue, the kit comprising: a needle insertable into an animal body tissue; a percutaneous lead operatively inserted into the needle, wherein the needle and the percutaneous lead are inserted into an insertion point of the animal body, whereby the needle is removable from the animal body tissue and the percutaneous lead is retained within the animal body; and a system comprising:
a coiled wire lead that may be inserted percutaneously into a portion of a body that is proximal to or will become or remain part of a residual limb or extremity following surgical amputation, wherein the thickness and a diameter of the coiled wire lead are appropriate for percutaneous insertion;
an electrode formed integrally on the lead, wherein the electrode is i) located inside the body when the system delivers stimulation, and ii) at a therapeutically effective distance from a portion of at least one nerve that will be transected or cut during the surgical amputation; and
an electrical stimulation device functionally connected to the lead to apply electrical stimulation through the at least one electrode to at least one nerve that will be transected or cut during the surgical amputation.

32. The kit of claim 31, wherein the electrical stimulation causes an area of comfortable sensations and the area of comfortable sensations is compared with the region of expected pain from the amputation surgery.

33. The kit of claim 31, further comprising a test needle.

34. The kit of claim 31, wherein the needle is an introducer needle.

35. The kit of claim 31, further comprising an anchor attached to the percutaneous lead, the anchor configured to operatively retain the percutaneous lead at the insertion point during withdrawal of the needle from the animal body.

36. A method to alleviate pain following an amputation surgery, the method comprising:
inserting a coiled wire lead with at least one electrode percutaneously into a body within a therapeutically effective distance from at least one nerve to be transected;
applying electrical stimulation through the at least one electrode to the nerve to be transected, thereby activating nerve fibers that innervate the region of pain expected from the amputation surgery in advance of the amputation surgery and causing an area of comfortable sensations in the body, said area of comfortable sensations being compared with an area affected by the at least one nerve innervating the region of pain expected from the amputation surgery.

37. The method of claim 36 wherein electrical stimulation is provided to the nerve to be transected prior to the amputation surgery.

38. The method of claim 36 wherein the electrical stimulation is provided to the nerve to be transected after said nerve is transected during the amputation surgery.

39. A method to alleviate pain following an amputation surgery, the method comprising:
inserting or instructing insertion of a coiled wire lead with at least one electrode percutaneously into a body within a therapeutically effective distance from at least one nerve to be transected;
wherein the at least one electrode is positioned outside of a region of pain expected from the amputation surgery;
delivering or instructing delivery of electrical stimulation through the at least one electrode to the nerve to be transected, thereby activating nerve fibers that innervate the region of pain expected from the amputation surgery in advance of the amputation surgery and causing an area of comfortable sensations in the body, said area of comfortable sensations being compared with an area affected by the at least one nerve innervating the region of pain expected from the amputation surgery.
